SEQUENCE LISTING (12 sequences)

SEQ ID NO: 1 - protein, 28 aa, synthetic construct
  REGION 1..28: note = Synthetic
LVKGGEAEAANIANAGLSAGPAANYLPA

SEQ ID NO: 2 - protein, 28 aa, synthetic construct
  REGION 1..28: note = Synthetic
LVNGGDDVAAAVNTAAQNNADALFVPPP

SEQ ID NO: 3 - protein, 28 aa, synthetic construct
  REGION 1..28: note = Synthetic
LSTAADMQGVVTDGMASGLDKDYLKPDD

SEQ ID NO: 4 - protein, 298 aa, Homo sapiens
MFSRAGVAGLSAWTLQPQWIQVRNMATLKDITRRLKSIKNIQKITKSMKMVAAAKYARAERELKPARIYGLGSLALYEKADIKGPEDKKKHLLIGVSSDRGLCGAIHSSIAKQMKSEVATLTAAGKEVMLVGIGDKIRGILYRTHSDQFLVAFKEVGRKPPTFGDASVIALELLNSGYEFDEGSIIFNKFRSVISYKTEEKPIFSLNTVASADSMSIYDDIDADVLQNYQEYNLANIIYYSLKESTTSEQSARMTAMDNASKNASEMIDKLTLTFNRTRQAVITKELIEIISGAAALD

SEQ ID NO: 5 - DNA, 21 bp, synthetic construct
  source 1..21: mol_type = other DNA
cagttcaccgtcaacctgtcc

SEQ ID NO: 6 - DNA, 19 bp, synthetic construct
  source 1..19: mol_type = other DNA
tggtgtgggcgatgacacg

SEQ ID NO: 7 - DNA, 20 bp, synthetic construct
  source 1..20: mol_type = other DNA
aacgggaagcttgtcatcaa

SEQ ID NO: 8 - DNA, 20 bp, synthetic construct
  source 1..20: mol_type = other DNA
tggactccacgacgtactca

SEQ ID NO: 9 - DNA, 16 bp, synthetic construct
  source 1..16: mol_type = other DNA
cgggaaggagaacctg

SEQ ID NO: 10 - DNA, 20 bp, synthetic construct
  source 1..20: mol_type = other DNA
gaccgctcggagtgtacttt

SEQ ID NO: 11 - DNA, 20 bp, synthetic construct
  source 1..20: mol_type = other DNA
actggaacggtgaaggtgac

SEQ ID NO: 12 - DNA, 20 bp, synthetic construct
  source 1..20: mol_type = other DNA
agagaagtggggtggctttt

(19) United States
(12) Patent Application Publication
(10) Pub. No.: US 2025/0179126 A1
(43) Pub. Date: Jun. 5, 2025

(54) A NOVEL PEPTIDE FROM A PHOTOSYNTHETIC BACERIUM DIRECTLY TARGETS MITOCHONDRIA TO TRIGGER APOPTOSIS IN ADVANCED PROSTATE CANCER CELLS

(71) Applicant: THE BOARD OF TRUSTEES OF THE UNIVERSITIES OF ILLINOIS, Urbana, IL (US)

(72) Inventors: Samer Naffouje, Urbana, IL (US); Tohru Yamada, Oak Park, IL (US); Tapas Das Gupta, River Forest, IL (US)

(21) Appl. No.: 18/840,242

(22) PCT Filed: Feb. 21, 2023
(86) PCT No.: PCT/US23/62920

§ 371 Date: Aug. 21, 2024

Related U.S. Application Data
(60) Provisional application No. 63/268,297, filed on Feb. 21, 2022.

(51) Int. Cl. C07K 14/195; A61K 35/74; A61K 38/00; A61P 35/00

(52) U.S. Cl. CPC C07K 14/195; A61K 35/74; A61P 35/00; A61K 38/00

(57) ABSTRACT

We describe the design of aurB, a novel, non-toxic cell-penetrating peptide derived from the bacterial electron transfer protein auracyanin B, and show that it induces caspase-mediated apoptotic cell death in prostate cancer cells through the mitochondria and independent of the tumor suppressor 1A protein p53. Further, a symbiotic relationship of P. aeruginosa and cancer cells is described.

### REFERENCE TO A PRIORITY DOCUMENT

This application claims benefit of U.S. Provisional Application No. 63/268,297 filed Feb. 21, 2022, which application is incorporated herein by reference in its entirety.

### REFERENCE TO A SEQUENCE LISTING

The instant application contains a Sequence Listing which has been filed electronically in ASCII format and is hereby incorporated by reference in its entirety. Said ASCII copy, created on Feb. 21, 2023, is named 46466-58.xml and is 13054 bytes in size.

### BACKGROUND OF THE INVENTION

Mitochondria play important roles in numerous biological processes, and their functions are often altered in cancer cells, making them attractive targets for cancer therapies. Traditional chemotherapeutic agents target mitochondria indirectly through upstream mitochondrial signaling pathways that are frequently impaired or inactivated in cancers (e.g., p53), especially those such as advanced prostate and ovarian cancer, which are typically lethal and exhibit a limited response to immune checkpoint inhibitors. Thus, there is a need for the development of novel therapies that directly target mitochondria in cancer cells.

Mitochondrial genome sequencing studies have shown proteins with homology to bacterial cupredoxins in the mitochondria of eukaryotes sunflowers plans (Helianthus). Moreover, in several types of bacteria, cupredoxins are involved in electron transfer in a variety of biological processes, including ATP production through nitrogen fixation and photosynthesis. For example, the cupredoxin protein azurin is involved in the electron transfer chain during the nitrogen fixation process in bacteria such as the opportunistic pathogen Pseudomonas aeruginosa. Azurin secreted by P. aeruginosa induces caspase-mediated apoptotic cell death through the complex formation with and activation of a tumor suppressor protein p53. Further, an azurin-derived cell penetrating peptide (also referred to as p28; NSC745104) also induced p53-mediated tumor growth inhibition in vivo.

Many advances in cancer treatment have been made by targeting strategies. Mitochondria are recognized as one of the most important targets for developing new therapeutic agents. Mitochondrial functions in eukaryotes include ATP generation and regulation of cell proliferation and death. Abnormal mitochondrial functions have long been associated with aberrant cancer cell proliferation. Mitochondrial ATP synthesis is often altered in cancer cells and has a crucial role in tumor growth and patient overall survival. Therefore, targeting mitochondria in cancer cells is a promising approach to developing new therapeutic agents. However, currently available agents indirectly target mitochondria by relying on upstream mitochondrial signaling pathways that are frequently impaired in cancer cells. Thus, new therapeutic agents that can directly target mitochondria in cancer cells would be ideal.

Microorganisms, particularly pathogenic bacteria, were used in the treatment of various types of human cancer over 100 years ago under the premise that a toxin produced by given pathogenic bacteria will inhibit the growth and spread of human cancer. With the increasing knowledge of the role of the microbiota and the increasing incidence of cancer, treatment options using this approach have been reevaluated.

Azurin is a 14 kDa periplasmic copper protein containing 128 amino acids (aa) that are found in several types of bacteria and blue-green algae. One of its known biological functions is as an electron transfer protein in anaerobic energy production via nitrogen fixation. In vitro studies have demonstrated that azurin induces apoptotic cell death in a variety of cancer cells, with minimal or no effect on their normal counterparts. Upon preferential entry into cancer cells, azurin binds to the tumor suppressor protein p53 and induces caspase-mediated apoptosis. Human xenotransplant studies in athymic mice showed that systemic administration of azurin inhibited tumor growth without significant adverse effects on the host. In addition, we previously identified the active peptide fragment of azurin (i.e., p28 or Azu28), and the efficacy of this 28-aa cell-penetrating peptide p28 has been extensively investigated both experimentally and clinically.

### SUMMARY OF THE INVENTION

Based on the uniqueness of auracyanins among cupredoxins and the known biological functions of cupredoxins, we discovered that these bacterial proteins might modulate human mitochondrial homeostasis by directly targeting mitochondria.

Here, we describe the design of aurB, a novel, non-toxic cell-penetrating peptide derived from the bacterial electron transfer protein auracyanin B and show that it induces caspase-mediated apoptotic cell death in prostate cancer cells through the mitochondria and independent of the tumor suppressor protein p53.

Further, we describe, P. aeruginosa has acquired abilities to both harm (parasitism) and benefit human health (mutualism) and adjusts its behavior depending on physiological/cellular conditions that can change depending on disease status. P. aeruginosa can benefit human health by suppressing and attacking malignant cells through secretion of the bacterial protein azurin in hosts harboring malignant cells. P. aeruginosa expressing azu (azurin) was found in tumors of primary melanoma and breast cancer patients who did not receive chemotherapy before specimen collection.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE SEQUENCE LISTING

SEQ ID NO: 1 is an auracyanin A peptide, aurA, corresponding to amino acids 62-89. LVK GGE AEA ANI ANA GLS AGP AAN YLPA (SEQ ID NO:1).

SEQ ID NO: 2 is an auracyanin B peptide, aurB, corresponding to amino acids 61-88. LVN GGD DVA AAV NTA AQN NAD ALF VPPP (SEQ ID NO:2).

SEQ ID NO: 3 is a p28 peptide corresponding to amino acids 50-77 of azurin. LST AAD MQG VVT DGM ASG LDK DYL KPDD (SEQ ID NO:3).

SEQ ID NO: 4 is an ATPG human ATP synthase subunit gamma, mitochondrial sequence. Met Phe Ser Arg Ala Gly Val Ala Gly Leu Ser Ala Trp Thr Leu Gln Pro Gln Trp Ile Gln Val Arg Asn Met Ala Thr Leu Lys Asp Ile Thr Arg Arg Leu Lys Ser Ile Lys Asn Ile Gln Lys Ile Thr Lys Ser Met Lys Met Val Ala Ala Ala Lys Tyr Ala Arg Ala Glu Arg Glu Leu Lys Pro Ala Arg Ile Tyr Gly Leu Gly Ser Leu Ala Leu Tyr Glu Lys Ala Asp Ile Lys Gly Pro Glu Asp Lys Lys Lys His Leu Leu Ile Gly Val Ser Ser Asp Arg Gly Leu Cys Gly Ala Ile His Ser Ser Ile Ala Lys Gln Met Lys Ser Glu Val Ala Thr Leu Thr Ala Ala Gly Lys Glu Val Met Leu Val Gly Ile Gly Asp Lys Ile Arg Gly Ile Leu Tyr Arg Thr His Ser Asp Gln Phe Leu Val Ala Phe Lys Glu Val Gly Arg Lys Pro Pro Thr Phe Gly Asp Ala Ser Val Ile Ala Leu Glu Leu Leu Asn Ser Gly Tyr Glu Phe Asp Glu Gly Ser Ile Ile Phe Asn Lys Phe Arg Ser Val Ile Ser Tyr Lys Thr Glu Glu Lys Pro Ile Phe Ser Leu Asn Thr Val Ala Ser Ala Asp Ser Met Ser Ile Tyr Asp Asp Ile Asp Ala Asp Val Leu Gln Asn Tyr Gln Glu Tyr Asn Leu Ala Asn Ile Ile Tyr Tyr Ser Leu Lys Glu Ser Thr Thr Ser Glu Gln Ser Ala Arg Met Thr Ala Met Asp Asn Ala Ser Lys Asn Ala Ser Glu Met Ile Asp Lys Leu Thr Leu Thr Phe Asn Arg Thr Arg Gln Ala Val Ile Thr Lys Glu Leu Ile Glu Ile Ile Ser Gly Ala Ala Ala Leu Asp(SEQ ID NO: 4).

SEQ ID NO: 5 is an azu-specific primer; 5′-CAGTTCACCGTCAACCTGTCC-3′(SEQ ID NO: 5).

SEQ ID NO: 6 is an azu-specific primer and 5′-TGGTGTGGGCGATGACACG-3′(SEQ ID NO: 6).

SEQ ID NO: 7 is a Human GAPDH primer was also amplified as a loading control; 5′-AACGGGAAG CTTGTCATCAA-3′(SEQ ID NO: 7).

SEQ ID NO: 8 is a Human GAPDH primer was also amplified as a loading control 5′-TGGACTCCACGACGTACTCA-3′(SEQ ID NO: 8).

SEQ ID NO: 9 is a primer sequence Aldolase A: forward 5′-CGG GAA GGA GAA CCT G-3′(SEQ ID NO: 9).

SEQ ID NO: 10 is a primer sequence Aldolase A reverse 5′-GAC CGC TCG GAG TGT ACT TT-3′(SEQ ID NO: 10).

SEQ ID NO: 11 is a primer sequence β-actin: forward 5′-ACT GGA ACG GTG AAG GTG AC-3′(SEQ ID NO: 11).

SEQ ID NO: 12 is a primer sequence β-actin:reverse 5′-AGA GAA GTG GGG TGG CTT TT-3′(SEQ ID NO: 12).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1I: Overall structures of auracyanins A and B from C. aurantiacus and azurin from P. aeruginosa. Ribbon diagrams and molecular surfaces of each protein (1A: auracyanin A, 1B: auracyanin B, 1C: azurin) were imaged by QuteMol and DeepView (Swiss Institute of Bioinformatics). Dotted boxes indicate the locations of the helical (red) peptides aurA, aurB and p28. Blue: copper. Hydrophobicity plots of aurA (1D), aurB (1E) and p28 (1F) were generated according to the hydrophobicity scale of Kyte and Doolittle. The polarity scores of aurA (1G), aurB (1H) and p28 (1I) were calculated according to the polarity propensity scale.

FIG. 2A-2B: Characteristics of the three proteins and their peptides. Primary sequences of mature proteins were used. The peptide sequences are described in the Methods section (2A). Multiple sequence alignment with ClustalO indicated that no amino acids were conserved among all three peptides (2B). aurA (auracyanin A aa 62-89): LVK GGE AEA ANI ANA GLS AGP AAN YLPA (SEQ ID NO:1), aurB (auracyanin B aa 61-88): LVN GGD DVA AAV NTA AQN NAD ALF VPPP (SEQ ID NO:2), and p28 (azurin aa 50-77): LST AAD MQG VVT DGM ASG LDK DYL KPDD (SEQ ID NO:3). “:” and “.” indicate conserved substitutions (between two of three peptides) and semi-conserved substitutions (among all three peptides), respectively.

FIG. 3A-3B: Anti-proliferative effects of aurA and aurB. Human cancer cells (2,000 cells/well) were incubated with different concentrations (5-100 μM) of aurA (3A) and aurB (3B) at 37° C. for 24 hr. Cell viability was detected by MTT assays, and the control (PBS-treated) cells were considered 100% viable. Mean±SE (N=3). *P<0.05, **P<0.01, ***P<0.001.

FIG. 4A-4D: Effect of peptides on prostate cancer cell lines with differences in p53 and AR expression status. MTT cell viability assays on prostate cancer cell lines (4A: LNCaP, 4B: DU145, 4C: PC3) and normal prostate cells (4D: CRL11611) were conducted in the presence of aurA, aurB, p28 or Ptxl for 24-72 hr. Control (PBS)-treated cells were considered to have 100% viability. Mean±SE (N=3).

FIG. 5: Effect of aurB on p53-null human cancer cells. The dose-dependent effect of aurB on PC3 prostate cancer, MDD2 breast cancer and SKOV3 ovarian cancer cells was measured by MTT assays. Mean±SE (N=3). Each cancer cell line was exposed to aurB at 0.1-100 μM for 24 hr.

FIG. 6A-6B: Cellular membrane toxicity assays. Lactate dehydrogenase (LDH) leakage assays were conducted on PC3 cells exposed to different concentrations (0.2-200 μM) of aurA (6A) or aurB (6B) at 37° C. Cells treated with lysis buffer were defined as having 100% LDH release. Mean±SE (N=3).

FIG. 7A-7D: Determination of apoptotic cell death. Prostate cancer cell lines (7A: LNCaP, 7B: DU145 and 7C: PC3) and normal prostate CRL11611 cells (7D) were exposed to the indicated agents for 48 hr and stained using apoptosis assay kits according to the manufacturer's instructions. The samples were analyzed by flow cytometry. Mean±SE (N=3).

FIG. 8A-8D: AurB localizes to the mitochondria and targets ATP synthase. 8A, TEM image of PC3 cells. Representative TEM micrograph of aurB-treated cells depicting the intraritochondrial localization of aurB-GNRs (arrowheads) and mitochondria](Mt) swelling. Scale bar: 600 nm. Mt: mitochondria. 8B-8D, Identification of aurB binding protein. Mitochondrial fractions of PC3 cells were incubated with biotin-labeled p28, aurA or aurB. Coomassie-stained SDS-PAGE gels are shown (8B). The protein band at ˜35 kDa (arrowhead) was identified as ATP5C using mass spectrometry analysis. The identified fragments are indicated in green underlined sequence (SEQ ID NO: 4)(8C). IB with an anti-ATP5C antibody confirmed that aurB physically bound to ATP5C (8D).

FIG. 9A-9B: Effect of aurB on the mitochondrial apoptosis signaling pathway. 9A, PC3 cancer cells were exposed to aurA or aurB at the indicated concentrations. Cells were stained by using JC-1 Mitochondrial Membrane Potential Assay Kits according to the manufacturer's instructions to measure mitochondrial membrane potential, and fluorescence was measured by flow cytometry. 9B, Caspase-3 is involved in aurB-induced apoptosis. The effects of the caspase-3 inhibitor Z-DEVD-FMK on PC3 cells exposed to 100 μM aurB were determined by MTT assays at 24 hr (circle) and 48 hr (square). The inhibitor was used at the indicated concentrations. Mean±SE (N=3).

FIG. 10A-10D: Inhibition of PC3 xenograft tumor growth. 10A, When the s.c. tumors reached ˜5 mm, athymic mice were randomized into control (PBS) and i.p. treatment groups. Mean±SE (N=5). *P<0.01. 10B, None of the treatments significantly altered the body weight of the animals. 10C, At the end of treatment, tumors were dissected and weighed. * P<0.01. 10D, In sections of tumors from the control group, there was a marked increase in Ki-67-positive proliferating cells (stained brown and counterstained with haematoxylin, ×40), and only a few apoptotic cells were observed, as determined by TUNEL and caspase-3 (Casp-3) staining. There was an apparent sharp reduction in Ki-67-positive proliferating cells and a higher number of TUNEL-positive apoptotic cells in the aurB-treated group compared to the control group. Similarly, Casp-3 expression in aurB-treated tumors was increased markedly compared with that in untreated controls.

FIG. 11A-11B: A model of the direct targeting of the mitochondrial energy production system. 11A: Electron transfer complexes and subunits of ATP synthase localize to the mitochondrial inner membrane and generate energy in eukaryotic cells. In the bacterium Chloroflexus, auracyanins A and B are both membrane-anchored electron transfer proteins, unlike azurin from P. aeruginosa. Auracyanin A is proposed to be transported to an outer membrane, and auracyanin B is tethered to the inner membrane. 11B: A phylogenetic tree constructed based on the DNA sequences of azurin, auracyanin A, auracyanin B, and ATP5C by using the NGPhylogeny.fr program.

FIG. 12: TEM image of PC3 cells exposed to aurA. Representative TEM micrograph of aurA-treated PC3 cells showed that aurA-GNRs (arrowheads) were found predominantly in large intracellular vacuoles but not in mitochondria. Scale bar: 600 nm. *: mitochondria.

FIG. 13A-13C: Serum azurin levels are elevated in cystic fibrosis patients: 13A. Patient demographics. Serum from cystic fibrosis (CF) patients and controls (N=50 each) with similar age ranges/median rages was used to measure serum azurin levels. 13B. Serum levels of azurin in CF patients (N=50) and controls (N=50) were determined by ELISA with a rabbit polyclonal anti-azurin antibody an anti-azurin antibody. For internal standards, purified azurin was used. 13C. The linear regression line of azurin level vs. CF patient age was plotted. R=0.596, P=0.0038. Statistical analysis (t-test) showed a significant difference in azurin levels between CF patients and healthy controls. ****: P<0.0001.

FIG. 14A-14D: Azurin secretion is stimulated by human cancer cells. 14A Induction of azurin secretion by P. aeruginosa in the presence of host cells. Human cells and P. aeruginosa were co-incubated for 30 min at a concentration of 500,000 human cells/ml and P. aeruginosa (Pa) at an optical density (OD)=0.3. Human cell lines derived from various tissues and tumors were used: melanoma Mel-2, congenital melanocytic nevus CMN, prostate cancer DU-145, normal prostate CRL-11611, ovarian cancer SK-OV3, normal ovary HOSE6-3, breast cancer MDA-MB-231, and normal breast MCF-10A. Secretion of azurin by P. aeruginosa into the culture supernatant was assessed by western blot analysis; the graph shows the observed band intensities determined by a densitometer UN-SCAN-IT gel version 5.1. The mean+SE values were calculated for skin, prostate, ovarian, and breast cell pairs. 14B-14C Azurin secretion is cancer cell dose-dependent. P. aeruginosa secretes higher levels of azurin in the presence of human breast cancer MDA-MB-231 (14B) and melanoma Mel-2 cells (14C) than in the presence of melanocytes (CMN) or non-malignant MCF-10A breast cells, in a dose-dependent manner. Cancer and normal/non-malignant cells were co-incubated with P. aeruginosa for 30 min at concentrations ranging from 0 to 2,000,000 cells/ml and P. aeruginosa at OD=0.3. Secretion of azurin by P. aeruginosa into the culture supernatant was assessed by western blot analysis; the graph shows the observed band intensities. The mean+SE values were calculated. 14D Soluble extracellular factors stimulate azurin secretion in a distance-dependent manner. Mel-2 and P. aeruginosa cells were separated by a permeable Transwell® insert membrane with a pore size of 0.4 μm. In this co-culture system, P. aeruginosa (OD=0.3) and Mel-2 cells (3,000,000 cells/ml) were separated at distances ranging from 2 mm to 12 mm and incubated for 30 min at 37° C. (inset figure: schematic of the co-culture system. Top: P. aeruginosa, bottom: Mel-2). Secretion of azurin by P. aeruginosa into the culture supernatant was assessed by western blot analysis; the graph shows the observed band intensities.

FIG. 15: Azurin transcription is induced upon co-incubation of P. aeruginosa with Mel-2 cells. P. aeruginosa and Mel-2 cells were indirectly co-incubated for 0 to 120 min, and azu transcription was assessed by real-time PCR. Analysis of ΔΔCt values was conducted for azu transcripts with normalization to rpoD transcripts. azu and rpoD mRNA was isolated at various time points of co-incubation of P. aeruginosa cells with Mel-2 cells. The data revealed a 2-fold increase in the azurin transcript level at the 30-minute time point. Mean+SD, *P<0.05, ***P<0.001 (ANOVA, vs. the control (rpoD).

FIG. 16: P. aeruginosa does not pass through the 0.4 μm filter membrane. Coming Transwell® polyester membrane cell culture plates and inserts (TC-treated, sterile 24 mm Transwell with 0.4 μm pores) were used for the assays. Various concentrations of P. aeruginosa in 0.5% MGM were incubated in the upper compartment (insert) at 37° C. After 30 min incubation, culture media from upper and lower (well) chambers were plated on LB agar plates and determined the colony-forming units (CFU/ml).

FIG. 17A-17C: Aldolase A secretion by melanoma cells is induced by exposure to azurin. Aldolase A is secreted by Mel-2 cells in the presence of P. aeruginosa. 17A. Mel-2 or CMN cells (3,000,000 cells/ml) and P. aeruginosa (OD=0.3) were co-incubated in the Transwell system, and culture supernatants were collected and subjected to SDS-PAGE followed by Coomassie staining. The Mel-2+P. aeruginosa sample demonstrated one additional band (at 40 kDa) compared to the Mel-2 sample. 17B. Thisprotein band was analysed by mass spectrometry, and the protein was identified as human aldolase A. 17C. The numbers circled in red indicate peptides that matched the aldolase A sequence. Based on the mass spectrometry data, Mascot software was used to identify protein/peptide sequences. Matched peptide sequences to human aldolase A from the mass spectrometry data are shown in red.

FIG. 18A-18G: Induction of aldolase A secretion by host cells in the presence of P. aeruginosa. 18A. Human host cells (cancer and normal) and P. aeruginosa were co-incubated for 30 min at a concentration of 500,000 human cells/ml with P. aeruginosa at OD=0.3. Aldolase secretion varied between the co-cultures with cancer and normal cells. Secretion of azurin and aldolase into the culture supernatant was assessed by western blot analysis; the graph shows the observed band intensities. 18B Correlation between the aldolase A and azurin levels in co-cultures of P. aeruginosa with cancer or normal cells. 18C Cancer cells (Mel-2 and MDA-MB-231) and P. aeruginosa (WT and azunull mutant) were co-incubated for 30 min at a concentration of 500,000 human cells/ml with P. aeruginosa at OD=0.3. Secretion of aldolase into the culture supernatant was assessed by western blot analysis. 18D P. aeruginosa (OD=0.3) was treated with purified aldolase A protein at concentrations of 1 nM, 10 nM, 100 nM, and 1 μM for 30 min. Treatment with 1 M aldolase A stimulated azurin secretion from P. aeruginosa, suggesting that aldolase A is a stimulatory factor for azurin secretion. Secretion of azurin by P. aeruginosa into the culture supernatant was assessed by western blot analysis; the graph shows the observed band intensities. MDA-MB-231 (18E) and Mel-2 (18F) cells were treated with purified azurin protein at concentrations of 100 nM, 10 μM, and 1 mM for 30 min. Secretion of aldolase A by cancer cells into the culture supernatant was assessed by western blot analysis; the graph shows the observed band intensities. P. aeruginosa (Pa only) did not show any signal as it did not secret any proteins that cross-react with anti-aldolase A antibody. 18G Aldolase A secretion in the presence of P. aeruginosa is distance-dependent. Mel-2 cells and P. aeruginosa were separated with a permeable Transwell® insert. In this co-culture system, P. aeruginosa (OD=0.3) and Mel-2 cells (3,000,000 cells/ml) were separated at distances ranging from 0 mm to 12 mm and incubated for 30 min at 37° C. Aldolase A secretion decreased as the distance between the two cell populations increased. Secretion of aldolase by Mel-2 cells into the culture supernatant was assessed by western blot analysis; the graph shows the observed band intensities.

FIG. 19A-19B: P. aeruginosa does not induce cytotoxicity in Mel-2 cells during co-incubation for 30 min. To confirm that the factors found in the extracellular environment of Mel-2 cells are released due to active secretion and not due to cytotoxicity, cell viability assays were conducted on Mel-2 cells co-incubated with P. aeruginosa. The mean+SEM values were calculated from (19A) cell count and (19B) MTT cell proliferation assay data, and no significant difference was observed between the viability of Mel-2 cells in monoculture or in co-culture with P. aeruginosa.

FIG. 20: Western blot analyses of azurin in WT and mutant P. aeruginosa. Cell lysates of wild type (WT) and azu gene null P. aeruginosa PAO1 were loaded on the 4-12% NuPAGE gels. Bacterial proteins in the gel were transferred to the nitrocellulose membrane. Rabbit anti-azurin antibody (1:5,000) and anti-GAPDH (1:5,000) antibodies were applied and HRP-conjugated secondary antibodies were used for band visualization.

FIG. 21: E. coli harbouring the P. aeruginosa azu gene (ECAzu) induces aldolase secretion from Mel-2 cells. Both P. aeruginosa (Pa) and ECAzu demonstrated azurin secretion in the presence of Mel-2 cells. However, compared to ECAzu, Pa elicited a greater than 4-fold increase in azurin secretion. E. coli (EC), ECAzu, and Pa stimulated aldolase secretion from Mel-2 cells. Compared to EC, ECAzu demonstrated a 2-fold increase and Pa demonstrated a 6-fold increase in aldolase secretion. Secretion of azurin and aldolase into the culture supernatant was assessed by western blot analysis. Mean±SEM.

FIG. 22A-22B: Azurin does not modulate the gene expression or intracellular levels of aldolase A. MDA-MB-231 and Mel-2 cells were treated with purified azurin protein at concentrations of 100 nM, 10 μM, and 1 mM for 30 min. Aldolase A gene expression in MDA-MB-231 (black bars) and Mel-2 (white bars) cells was determined by RT-PCR (22A). N.S.: not significant. Mean±SEM. 22B: Intracellular levels of aldolase A were assessed by western blot analysis with an anti-aldolase A antibody. GAPDH was used as a loading control.

FIG. 23A-23B: siRNA-induced silencing of aldolase A (23A) and MUC1 (23B) genes in MDA-MB-231 (left) and Mel2 (right) cells. SMARTpool human ALDOA, MUC1 and non-targeting siRNA pool (Ctrl) were used as siRNA targeting aldolase A, MUCI and control (Ctrl), respectively. After 48 h of transfection, whole cell lysates (30 Apg/lane) were loaded on 4-12% NuPAGE gels. Proteins in the gels were transferred to the nitrocellulose membranes. Anti-aldolase, MUC1, and anti-GAPDH antibodies were applied, and HRP-conjugated secondary antibodies were used for band visualization.

FIG. 24A-24E: 24A—Adherence assays were performed essentially as previously described38. Monolayer MDA-MB-231 (red) and Mel-2 (blue) cells were compared to siRNA-induced silencing of aldolase gene in the cancer cell lines (+) when they were co-incubated with P. aeruginosa for 2 h. To assess total cell association, monolayers were washed to remove unbound P. aeruginosa and were then disrupted and homogenized in 0.10% saponin/PBS. P. aeruginosa cells were counted by serial dilution of the homogenized suspensions and subsequent determination of colony-forming units (CFU) by plating on LB agar. Control (−) expressed as 100%. Mean±SE, *P<0.05. 24B—Monolayer MDA-MB-231 (red) and Mel-2 (blue) cells were co-incubated with P. aeruginosa in the presence or absence of exogenous aldolase A for 2 h. Similar to above, total P. aeruginosa association on cancer cells were counted by plating on LB agar. Control (0 μM aldolase A) expressed as 100%. 24C—Monolayer MDA-MB-231 (red) and Mel-2 (blue) cells were compared to siRNA-induced silencing of MUC1 genes in the cancer cell lines (+) when they were co-incubated with P. aeruginosa in the presence or absence of exogenous aldolase A at 200 nM for 2 h. Total P. aeruginosa association on cancer cells were counted by plating on LB agar. *P<0.05, NS: not significant. Azurin secretion is P. aeruginosa dose-dependent. MDA-MB-231 (24D) and Mel-2 (24E) cells (500,000 cells/ml) were co-incubated with P. aeruginosa at concentrations corresponding to an OD ranging from 0.0 to 0.6. Secretion of azurin by P. aeruginosa into the culture supernatant was assessed by western blot analysis; the graph shows the observed band intensities. The mean+SE values were calculated.

FIG. 25: Demographics of patients with melanoma and breast cancer. Primary and metastatic tumours from patients with similar age ranges and median ages were used.

FIG. 26A-26H: Azurin-producing P. aeruginosa in human tumors. The azurin-encoding gene (azu) was detected in tumors from patients with breast cancer and melanoma. It was amplified by PCR with P. aeruginosa azu-specific primers and confirmed by DNA sequencing. The amplified PCR product, as a single band, was sequenced and showed 100% identity to P. aeruginosa azu. In melanoma (26A), 27.6% (8 of 29) of primary tumors and 5.9% (2 of 34) of metastatic tumors were azu positive (P<0.05). In breast cancer (26B), 22.7% (5 of 22) of primary tumors and 14.3% (2 of 14) of metastatic tumors were azu positive (P>0.05). In addition, frozen tumor samples were processed for immunogold transmission electron microscopy (TEM) by using anti-P. aeruginosa and anti-azurin antibodies. TEM images of uranyl acetate-stained sections (arrowheads) showed the intracellular localization of P. aeruginosa (26C) and its product azurin (26D) in human melanoma sections that were azu positive by PCR. Magnification: ×3000. 26E, 26F-Patients with azu-positive tumors displayed increased survival. Survival analysis of patients with azu-positive vs. azu-negative primary melanoma (26E, P<0.01) and primary breast cancer (26F, P<0.05) tumors. 26G—Hemizvgous MMTV-PyMT transgenic mice were injected with either PBS control or 2.5 mg/kg azurin (intraperitoneally, 3× weekly) for 2 months. The mean+SE values of tumor volume were calculated. h Tumor-free survival curve was generated with the PBS control and 2.5 mg/kg azurin groups of MMTV-PyMT transgenic mice. Median tumor-free survival of the PBS control and azurin-treatment groups was 67 and 72 days, respectively. P=0.015

FIG. 27A-27C: Melanoma specimen sections contain P. aeruginosa and azurin. 27A-H&E staining of human melanoma that was imaged by TEM in FIG. 26. The H&E-stained sections (20× magnification) confirmed a malignant tumor with high cellular atypia. In approximately 20% of tumor cells, the cytoplasm was stained brown due to the expression of melanin. TEM images showed the intracellular localization of azurin (27B, 6,300×; 27C, 1,000×) in the human melanoma sections (arrowheads).

FIG. 28: Invasive oestrogen receptor and progesterone receptor-negative breast cancer cells induce the highest level of azurin secretion by P. aeruginosa. P. aeruginosa secretes higher levels of azurin in the presence of highly invasive MDA-MB-231 (ER−, PR−, Her2−) human breast cancer cells than in the presence of non-invasive T-47D (ER+, PR+, Her2−) human breast cancer cells or the MCF-10A normal breast cell line. Cancer [highly invasive MDA-MB-231 (ER−, PR−, Her2−) human breast cancer cells and non-invasive T-47D (ER+, PR+, Her2−) human breast cancer cells] and normal/non-malignant cells (MCF-10A cells) were co-incubated with P. aeruginosa for 30 min. Azurin secretion by P. aeruginosa into the culture supernatant was assessed by western blot analysis; the graph shows the observed band intensities. Mean±SEM.

FIG. 29A-29C: Effect of azurin from P. aeruginosa in a transgenic animal model. 29A. Near-infrared red (NIR) dye-conjugated azurin or PBS was injected i.v. into transgenic mice that spontaneously develop mammary tumors. Twenty-four hours after azurin injection, mice were imaged with a photodynamic eye (PDE) system (left: photographic image, right: NIR fluorescence image at 800 nm). 29B. At the end of the study, the tumors were resected. The tumour weight of azurin-treated mice was significantly lower than that of control mice (** P<0.01), but the body weight did not differ (29C).

FIG. 30: A model of the P. aeruginosa-cancer interaction. Aldolase A secretion in response to the bacterial protein azurin a beneficial anti-cancer activity of bacteria.

### DETAILED DESCRIPTION OF THE INVENTION

### Definitions

As used in the specification and the claims, the singular forms “a,” “an,” and “the” include plural referents unless the context clearly dictates otherwise.

As used herein, the term “cell” includes either the singular or the plural of the term. The terms “isolated”, “purified” or “biologically pure” refer to material which is substantially or essentially free from components which normally accompany material as it is found in its native state. The term “heterologous DNA,” “heterologous nucleic acid sequence,” or “exogenous” and the like as used herein refers to a nucleic acid sequence wherein at least one of the following is true: (a) the sequence of nucleic acids foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such, as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s), “g” means gram(s), “μg” or “ug” means microgram(s) and “ng” means nanogram(s), “PCR” means polymerase chain reaction, “kDa” means kilodaltons, “g” means the gravitation constant, “bp” means base pair(s), “kbp” means kilobase pair(s), “% w/v” means weight/volume percent, “% v/v” means volume/volume percent, “rpm” means revolutions per minute, “HPLC” means high performance liquid chromatography, and “GC” means gas chromatography.

It is understood that “aurA” and “aurB” may refer to any molecule having a peptide sequence with substantial similarity to SEQ ID NOs: 1 or 2. It is appreciated that any polypeptide that includes partially or wholly SEQ ID NOs: 1 or 2, or is at least 96% identical to p28 is usable as a probe in the methods described herein. That is, if a single amino acid of aurA, aurB, or p28 is altered via substitution with a different amino acid, that new sequence would be 96% identical to SEQ ID NOs: 1 or 2. Likewise is a single amino acid is added to the p28 sequence, would result in a sequence that is 96% identical to SEQ ID NOs: 1 or 2.

### Overview

Because mitochondria are descended from bacterial endosymbionts, some protein characteristics are shared between mitochondria and extant bacteria that allow bacterial proteins to alter mitochondrial functions. Consistent with this model, we observed that aurB induced apoptosis by targeting the mitochondrial ATP synthase and modulating mitochondrial apoptosis pathways in cancer cells but not healthy cells. In this study, we developed new agents from such bacterial proteins that can directly target and modulate the mitochondrial signaling pathways that are aberrantly expressed in cancers. Collectively, our findings indicate that bacterial electron transfer proteins represent a significant source of novel therapeutic agents for directly targeting the mitochondrial energy production system in cancer cells.

Here, we describe the design of aurB, a novel, non-toxic cell-penetrating peptide derived from the bacterial electron transfer protein auracyanin B, and show that it induces caspase-mediated apoptotic cell death in prostate cancer cells through the mitochondria and independent of the tumor suppressor protein p53.

We identified a novel, non-toxic aurB peptide derived from a bacterial photosynthetic protein that targets mitochondria and induces caspase-mediated apoptotic cancer cell death independent of p53, demonstrating its potential therapeutic value in directly targeting mitochondria in cancer cells. mitochondria in cancer cells are structurally and functionally different from those in normal cells. Many conventional agents target signaling pathways that lie upstream of mitochondria and converge on these organelles to induce cell death, suggesting that the development of potential anticancer agents that directly target energy production machinery in mitochondria is a logical approach. To the best of our knowledge, this is the first study to show that bacterial photosynthetic proteins can be significant sources for immune-independent drug development directly targeting the mitochondrial energy production system. This study provides an important reference for the development of novel therapeutic agents through modulation of mitochondrial homeostasis.

We have investigated the therapeutic role of microbes, such as the opportunistic pathogen Pseudomonas aeruginosa, in the management of human cancers. Although P. aeruginosa is a major pathogen in cystic fibrosis (CF) causing significant morbidity and mortality, CF patients have a lower incidence of melanoma and breast cancer than non-CF patients. When supernatants of P. aeruginosa culture medium were co-incubated with tumor-derived J774A.1 macrophages, they induced apoptosis in a dose-dependent manner in J774A.1. The supernatants of the culture medium contained a high concentration of the P. aeruginosa-secreted redox protein azurin.

Here, we further show and describe that P. aeruginosa upregulated azurin secretion in response to increasing numbers of and proximity to cancer cells. Conversely, cancer cells upregulated aldolase A secretion in response to increasing proximity to P. aeruginosa, which also correlated with enhanced P. aeruginosa adherence to cancer cells. Additionally, we show that cancer patients had detectable P. aeruginosa and azurin in their tumors and exhibited increased overall survival when they did, and that azurin administration reduced tumor growth in transgenic mice. Our results indicate host-bacterial symbiotic mutualism acting as a diverse adjunct to the host defense system via inter-kingdom communication mediated by the evolutionarily conserved proteins azurin and human aldolase A. This improved understanding of the symbiotic relationship of bacteria with humans indicates the potential contribution to tumor homeostasis.

We have demonstrated the interaction of P. aeruginosa with human cancer cells and its role in tumor homeostasis. Besides highlighting the importance of this specific bacterial-cancer interaction, we show for the first time the bidirectional regulation of bacterial-cancer communication in relation to the potential for P. aeruginosa-secreted azurin to inhibit tumor growth.

In some aspects, the invention describes an auracyanin peptide that modulates mitochondrial mediated apoptotic pathways within a eukaryotic cell. Modulate may include increasing or decreasing any pathway typically found in a mitochondria. Of particular interest as pathways that result in stimulation of a pathway leading to cell death. Of particular interest is activation of a capase-dependent apoptic pathway within the mictochondria of a eukaryotic cell. In some aspects, the auracyanin peptide is 20-35 amino acids in length. In some aspects, the peptide is 28 amino acids in length. In some aspects, the peptide forms α-helical and β-sheet motifs in solution. In some aspects, the peptide is a auracyanin obtained from a C. aurantiacus microorganism. In some aspects, the peptide is amino acids 62-89 of an auracyanin A protein. In some aspects, the peptide is amino acids aa 61-81 of an auracyanin B protein. In some aspects, the peptide localizes to a mitochondrial membrane of the eukaryotic cell and binding to the peptide to a gamma subunit of a human mitochondrial ATP synthase protein occurs. In some aspects, the peptide targets mitochondrial mediated apoptotic pathways within cancer cells but not healthy cells. In some aspects, the peptide has a sequence that is 96% identical to SEQ ID NO: 1 or 2. In some aspects, the peptide has a sequence that is SEQ ID NO: 1 or 2. In some aspects, the peptide has a pI of about 3.42 and a molecular weight of about 2400-2700 Da. In some aspects, the auracyanin peptide is non-toxic to the eukaryotic cell and penetrates the eukaryotic cell. In some aspects, the peptide produces mitochondrial membrane potential loss in a dose dependent manner.

In some aspects, a method of modulating mitochondrial mediated apoptotic pathways within a eukaryotic cell is described. The method may include a step of administering to a eukaryotic cell a therapeutically effective amount of an auracyanin peptide, so that the auracyanin peptide induces produces mitochondrial membrane potential loss causing the induction of a caspase-mediated apoptotic pathway. In some aspects, the auracyanin peptide induces caspase-mediated apoptotic cell death in cancer cells but not healthy cells. In some aspects, the cell death induced by the auracyanin peptide is independent of a tumor suppressor protein p53 pathway. In some aspects, the auracyanin peptide is amino acids aa 61-81 of an auracyanin B protein. In some aspects, a method of administering to a subject in need of treatment for a cancer a therapeutically effective amount of an auracyanin peptide is described. The method may include steps of providing an amount of auracyanin peptide effective to enhance mitochondrial mediated apoptotic pathways within a cancer cell in a pharmaceutically acceptable form and administering the effective amount of auracyanin peptide to the subject. By practice of this method, the auracyanin peptide will induce caspase-mediated apoptotic pathways in cancer cells. In some aspects, the auracyanin peptide applied in this method may be amino acids aa 61-81 of an auracyanin B protein.

It will be appreciated that while in some aspects the invention describes a non-toxic cell permeable peptide and application there of to a cell or subject, the administration of the auracyanin peptide may occur by other means. That is, for example, a plasmid or other nucleic acid delivery vehicle comprising a nucleic acid encoding the peptide may be administered to the cell or subject to provide expression of the auracyanin peptide within the cell or subject.

### EXAMPLES

### Example 1: Characterization of Peptide-Based Agents from C. Aurantiacus Proteins

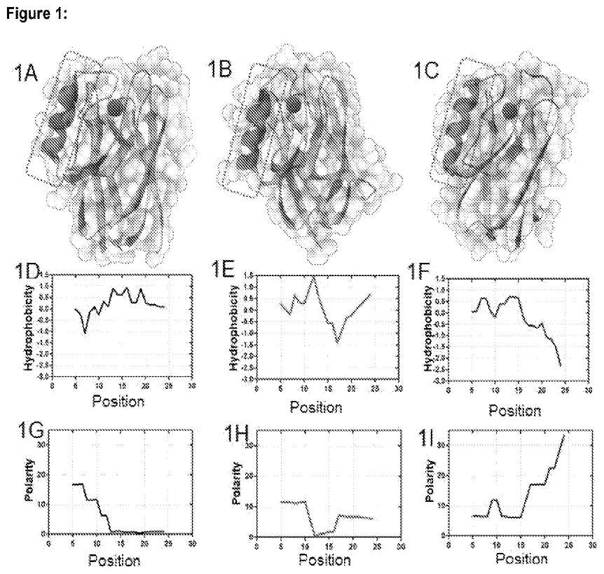

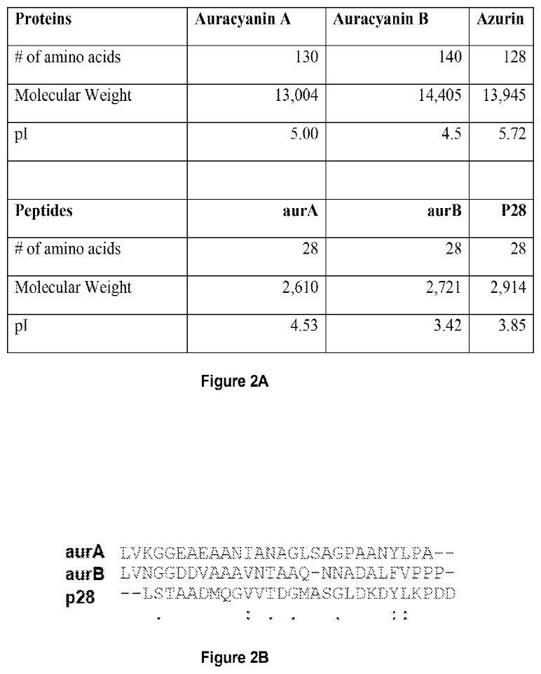

C. aurantiacus produces at least two distinct forms of auracyanins. Both the auracyanin A protein (13.9 kDa, 139 aa) and the auracyanin B protein (14.4 kDa, 140 aa) have a molecular core structure of two β-sandwich domains formed by eight polypeptide strands in a typical cupredoxin fold (FIG. 1A-1C). In previous work, we designed and identified the cell-penetrating peptide p28 from P. aeruginosa azurin, which forms α-helical and β-sheet motifs with random coils in solution. Here, with a similar approach, we designed peptide fragments from mainly helical motifs in auracyanins A and B. These fragments, aurA (aa 62 to 89 of auracyanin A) and aurB (aa 61 to 88 of auracyanin B), are highly water-soluble, 28-aa linear peptides with molecular weights of 2,610 and 2,721 Da, respectively. These two peptides and p28 are all anionic peptides, but the pI value of aurB is closer to that of p28 (FIG. 2A). Multiple sequence alignment showed that there was very little amino acid conservation among the three peptides (FIG. 2B), and hydrophobicity/polarity plots (FIG. 1D-1T) indicated that the polarity of aurA at the middle-C-terminal region is quite low compared to that of p28, suggesting that the biological functions of these three peptides would likely vary due to their different chemical characteristics, despite their similar secondary structures (FIG. 1).

### Example 2: Anti-Proliferative Effects of aurA and aurB on Cancer Cell Lines

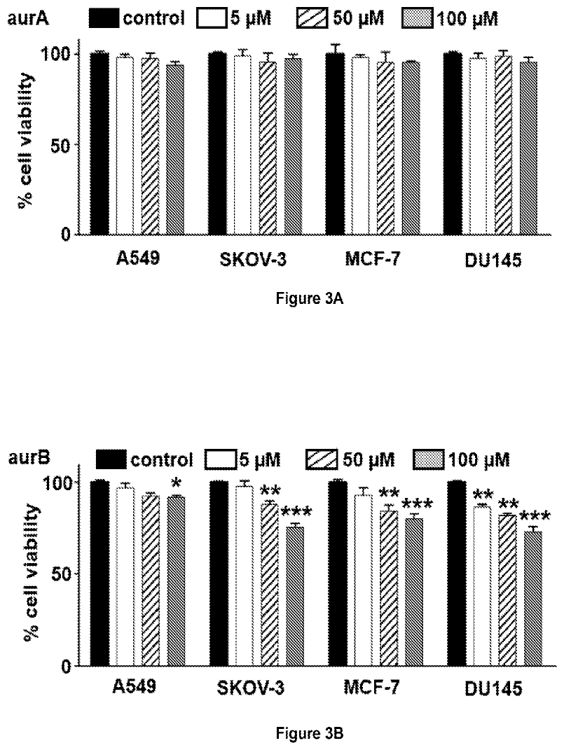

Given that p28 acts via a p53-dependent mechanism in cancer cells, we investigated the anti-proliferative effects of aurA and aurB on a variety of cancer cell lines with different p53 statuses in vitro. AurA treatment showed little effect at any concentration tested in human cancer cells (FIG. 3A). In contrast, aurB exposure showed clear dose-dependent effects on ovarian (SKOV-3), breast (MCF-7) and prostate (DU145) cancer cell lines, regardless of p53 expression status (FIG. 3B).

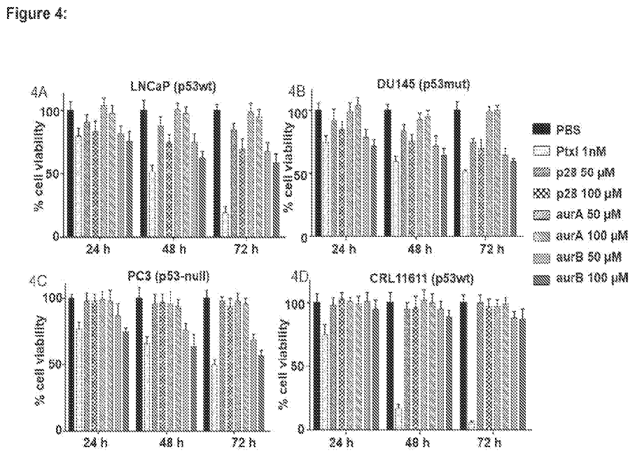

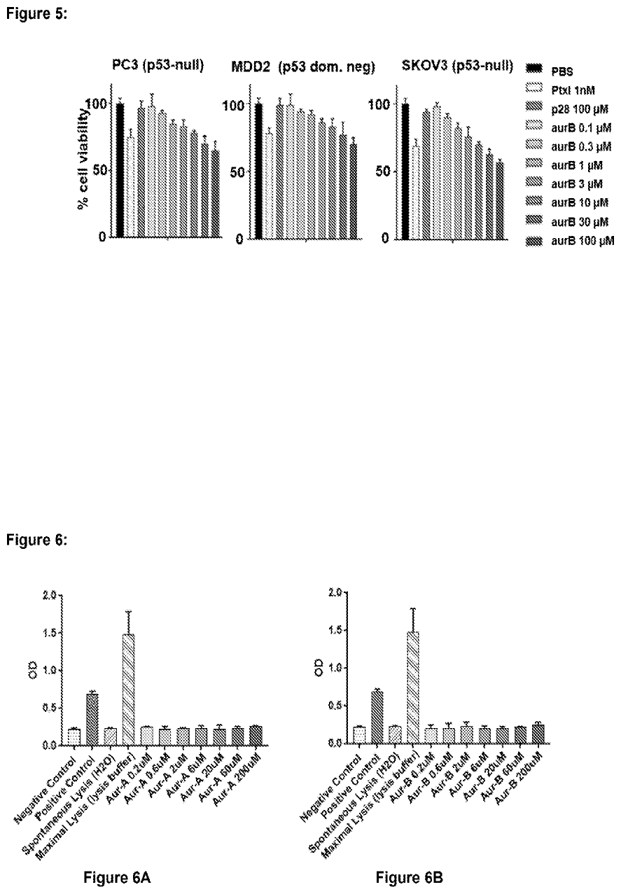

Although targeting the androgen receptor (AR) axis has been one of the most successful therapeutic approaches, ˜20% of prostate cancer patients develop castration-resistant prostate cancer (CRPC) stage within five years of follow-up during androgen deprivation therapy. CRPC has a poor prognosis and impairs quality of life. Additionally, current immunotherapy has shown limited efficacy for advanced prostate cancer, unlike for melanoma or lung cancer. Therefore, our observation that aurB significantly decreased the proliferation of an AR-negative DUl145 prostate cancer cell line prompted us to assess the effects of aurB, aurA, p28, and paclitaxel (Ptxl; one of the current standard drugs for prostate cancer) on additional prostate cancer cell lines with different molecular signatures. As expected, exposure to 1 nM paclitaxel significantly induced time-dependent cytotoxicity in the prostate cancer metastasis lines LNCaP (derived from left supraclavicular lymph node metastasis, p53 wild-type, AR+), DU145 (derived from central nervous system metastasis, heterozygous p53-mutant P223L and V274F, AR−) and PC3 (derived from bone metastasis, p53-null, AR−), as well as the normal prostate cell line CRL11611 (p53+, AR+) (FIG. 4A-4D). p28 also exhibited a dose-dependent effect on p53-expressing prostate cancer cells (LNCaP and DU145) but not p53-null PC3 cells or normal CRL11611 cells, confirming our earlier report. AurA was not active against these prostate cancer cells, consistent with the results shown in FIG. 5. In sharp contrast, aurB significantly induced dose-dependent cytotoxic effects on all three prostate cancer cell lines at an ˜50% inhibition rate at 100 μM for 72 hr (FIG. 4A-4C). However, this effect of aurB was not evident in normal prostate cells (FIG. 4D). Moreover, the effect induced by aurB on p53-inactive cancer cell lines, including MDD2 breast cancer (p53 dominant negative) and p53-null SKOV3 ovarian and PC3 prostate cancer cells, was dose-dependent (FIG. 5). Taken together, these data indicate that the cytotoxic effect of aurB against cancer cells was independent of p53 status, AR expression and cell type.

We demonstrated the anti-proliferative activity of aurB in human cancer cells with various histological types, including a set of prostate cancer cell lines widely used in therapeutic research. Importantly, aurB was effective against p53-WT, p53-mutant and p53-null lines. The fact that the tumor suppressor p53 is the most frequently mutated gene in cancer 53 and that these mutations typically alter p53 activity depends on mutation sites within the gene indicates that p53 is a relevant therapeutic target for cancer drug development. There have been intense efforts to target mutant p53 reactivation in cancer, and several compounds have been identified. However, this promising approach does not apply to p53-null cancer cells, suggesting that different therapeutic strategies to treat p53-null cancer cells are needed. Moreover, p53-null PC3 cells, but not LNCaP cells, are considered small cell neuroendocrine carcinomas that do not form glands and are negative for AR and prostate-specific antigen (PSA). Neuroendocrine prostate cancer is extremely aggressive, does not respond to hormonal therapy and is characterized by a relatively ‘cold’ tumor immune microenvironment, similar to other metastatic prostate cancers. Since the five-year survival rate of metastatic and advanced prostate cancer in the US is 31%, as opposed to nearly 100% for local prostate cancer, a new therapeutic option needs to be developed to improve survival.

### Example 3: Cytotoxic Effect of aurB is Due to Cellular Membrane Damage

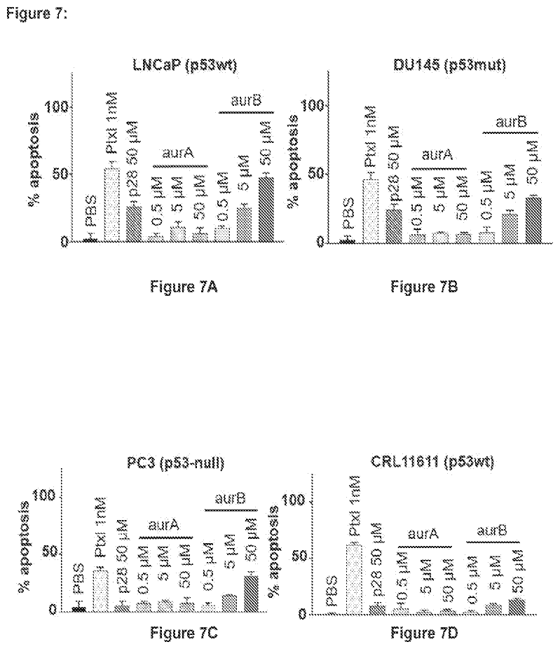

Many antimicrobial peptides induce their cytotoxic effects by disrupting the cellular membrane. To investigate whether the cytotoxic effect of aurB is due to cellular membrane damage, the membrane toxicity assays were performed. Neither aurA nor aurB induced cellular membrane toxicity at any concentrations (FIG. 6). We therefore examined apoptotic cell death in prostate cancer cell lines exposed to p28, aurA, aurB, and Ptxl (Paxitel) by flow cytometric analysis. In all cell lines, including normal cells, 1 nM Ptxl induced apoptotic cell death and aurA showed very little effect (FIG. 7A-7D). Additionally, p28 induced apoptotic cell death in LNCaP and DU145 cells but not PC3 and CRL11611 cells. Treatment with aurB induced significant dose-dependent apoptosis in all prostate cancer cell lines but less extent in normal prostate cells (FIG. 7A-7D). Importantly, although p53 and AR play significant roles in the induction of apoptosis, aurB can induce apoptosis in both p53-null and AR-negative cancer cells, suggesting that its mode of action is p53/AR-independent.

Apoptotic programmed cell death occurs in multicellular organisms, and mitochondria are particularly different from other subcellular organelles, as they play crucial roles in cellular energy metabolism and p53-dependent and p53-independent regulation of programmed cell death. Given that aurB is derived from a bacterial electron transport chain protein, we hypothesized that aurB might have an affinity for mitochondria as aurB was identified from auracyanin B, which is involved in electron transport chain processes for energy production in C. aurantiacus.

In this study, we compared the effects of aurA and aurB with those of Ptxl, which is one of the current standard chemotherapeutic agents for patients with prostate, breast and ovarian cancer. Ptxl was most toxic in the normal prostate cell line (CRL11611), and p53 mutant cell lines (DU145 and PC3) were less susceptible to Ptxl treatment than p53 wild-type and androgen-sensitive LNCaP cells. In contrast, all three prostate cancer cell lines tested were more sensitive than the normal prostate cell line to aurB. The results also confirmed that aurB significantly induced apoptotic cell death in prostate cancer cells compared with their normal counterparts (FIG. 7). It has been reported that overall mitochondrial mass is increased in carcinoma tissues compared to benign prostate tissues for all Gleason grades, which may be one reason why the effects of aurB on prostate cancer cells are significantly greater than those on normal prostate cells (FIG. 4, 7). Moreover, in cancers, high levels of mitochondrial ATP synthase complex V components, including ATP5C, are a significant risk factor (e.g., in terms of overall survival), suggesting that this complex is a promising druggable target.

### Example 4: Localization of aurB in PC3 Prostate Cancer Cells by Transmission Electron Microscopy (TEM)

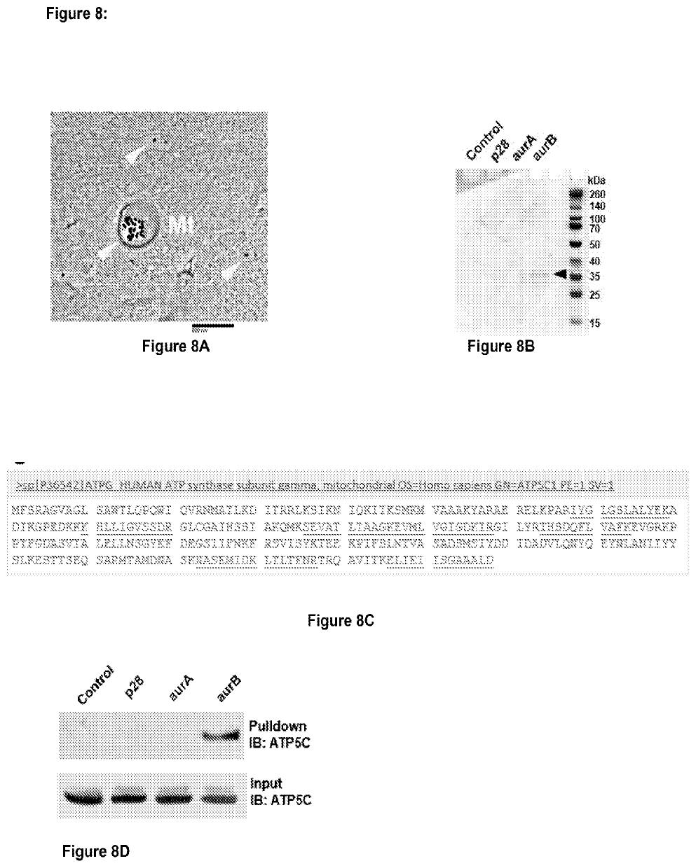

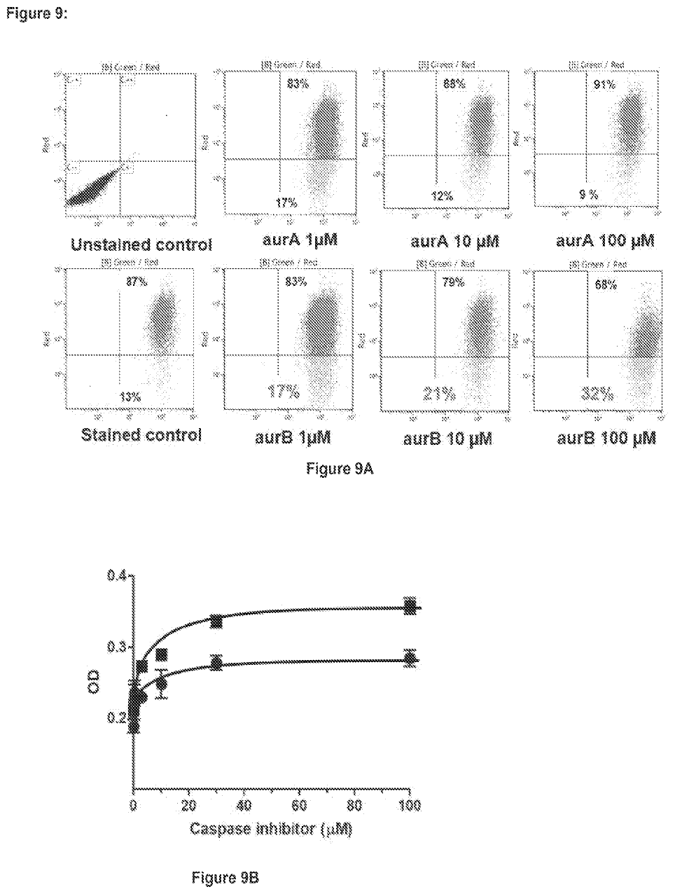

We first observed the localization of aurB in PC3 prostate cancer cells by transmission electron microscopy (TEM). AurB was conjugated to non-spherical gold nanorods for labelling (GNRs, diameter 25 nm×length 73 nm); relatively large GNRs were used because GNRs smaller than 15 nm in diameter can enter the tumor tissue by themselves. GNR-conjugated aurB was evidently internalized by PC3 cells and was localized in mitochondria (FIG. 8A). In contrast, GNR-conjugated aurA was internalized by PC3 cells and was found predominantly in large intracellular vacuoles but not in mitochondria (FIG. 9). Pull-down assays with biotin-labeled p28, aurA or aurB revealed that aurB pulled down an endogenous protein with a molecular weight of ˜35 kDa, but p28 and aurA did not (FIG. 8B). Mass spectrometry analysis of the 35 kDa protein band and immunoblotting (IB) identified the binding partner of aurB as the mitochondrial ATP synthase gamma subunit (ATP5C) (FIG. 8C-8D). Further, phylogenetic analysis to determine the evolutionary relationships among these three cupredoxins and ATP5C showed that auracyanin B is closely related to ATP5C (FIG. 8E), at least, in our phylogenetic analysis based on their gene sequences. Together, these findings suggest that the intramitochondrial localization of aurB occurs through binding to mitochondrial ATP5C and leads to mitochondrial swelling (FIG. 8A).

### Example 5: Mitochondrial Membrane Potential in PC3 Cells Treated with Different Compounds

Since the alteration of mitochondrial morphology is generally associated with mitochondrial membrane potential (Aym) loss and the caspase activation, we examined the mitochondrial membrane potential in PC3 cells treated with different compounds using a specific fluorescent probe (JC-1). AurA exposure did not impact AVm in PC3 cells, whereas aurB exposure dose-dependently increased AWm in these cells (1 μM: 17%, 10 μM: 21%, 100 μM: 32%) (FIG. 9A). Moreover, it has been reported that mitochondrial localization of apoptotic regulators causes Δψₘ loss, which triggers the activation of caspases, crucial mediators of apoptotic cell death. Since caspase-3 is a key mediator of mitochondrial apoptosis, the effects of Z-DEVD-FMK, a cell-permeable inhibitor that irreversibly binds to the catalytic site of caspase-3, were assessed to determine whether caspases mediated aurB-induced apoptosis. The caspase-3 inhibitor significantly blocked the aurB-induced cytotoxic/apoptotic effect in PC3 cells in a dose-dependent manner (FIG. 9B). These in vitro results demonstrated that aurB localized to mitochondria and induced apoptotic prostate cancer cell death through Δψₘ loss and caspase-3 activation independent of p53 and AR.

### Example 6: Effects of aurB Observed In Vivo

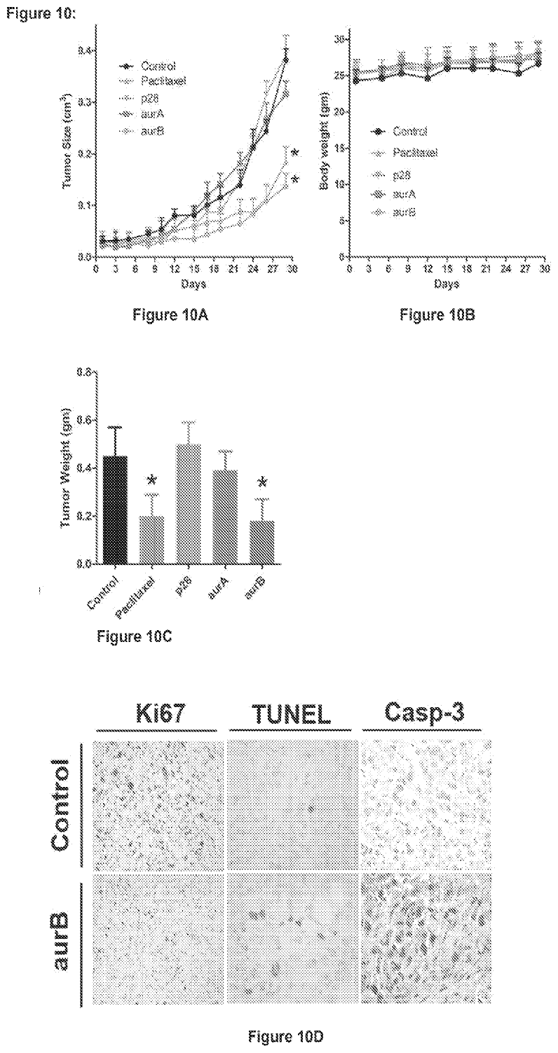

To investigate whether the effects of aurB observed in vitro can translate into in vivo, an athymic mouse xenograft model was use. In mice bearing PC3 xenograft tumors, aurB significantly inhibited (P<0.01) the growth of PC3 tumors over the course of four weeks of i.p. treatment (FIG. 10A) without inducing either a behavioral change or a loss in body weight (FIG. 10B). At the end of treatment, aurB and Ptxl treatment had inhibited tumor growth by ˜65% and 52% (vs PBS control), respectively. In contrast, p28 did not exhibit an antitumor effect on p53-null PC3 xenografts, consistent with the in vitro data (FIG. 4, 5, 7). AurB significantly decreased tumor weight, consistent with the tumor growth data (FIG. 10A, 10C). The histological analyses of tumors showed that proliferating cells stained for Ki-67 were randomly distributed among the tumor parenchyma of control animals (FIG. 10D). However, treatment with aurB substantially reduce the number of proliferating Ki-67-positive cells (FIG. 10D). To determine the apoptotic effect of aurB in vivo, caspase-3 staining and TUNEL assays, which detect DNA fragmentation, were employed. The tumors of control animals included a few TUNEL-positive apoptotic cells, but the number of apoptotic cells was increased in aurB-treated animals (FIG. 10D). A substantial increase in caspase-3-positive cancer cells was found in the tumors of aurB-treated animals compared to those of control animals (FIG. 10D). These data confirmed the in vitro data presented above and suggest that aurB suppresses tumor growth by inducing caspase-mediated apoptosis.

### Example 7: Bacterium and Auracyanins

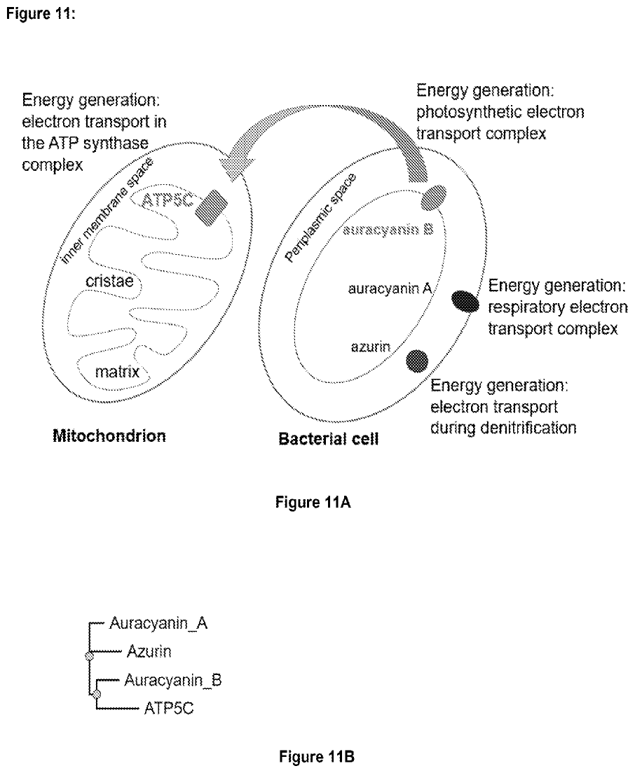

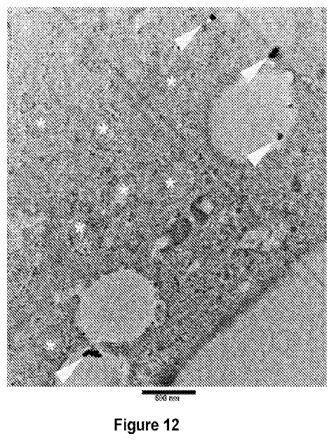

C. aurantiacus can be commonly found in bacterial mats of hot springs with a temperature range between 45 and 70° C. and a pH range between 6.5 and 9.0 and grow phototrophically under anaerobic conditions or chemotrophically under aerobic and dark conditions. The presence of multiple metabolic pathways and the ability to grow under such extreme conditions suggests that Chloroflexus has evolved under various environments. Based on 16S rRNA analysis, Chloroflexii species are the earliest branching bacteria capable of photosynthesis, and C. aurantiacus has long been regarded as a key organism to resolve the obscurity of the origin and early evolution of photosynthesis. In general, prokaryotes, including C. aurantiacus, can use a wide range of electron donors and acceptors during energy production and may have alternative complexes performing the same catalytic reactions as the mitochondrial complexes. Mitochondria have key roles in energy production and apoptosis in eukaryotic cells, and they are also considered descendants of endosymbiotic bacteria. In the bacterium Chloroflexus, similar to many mitochondrial proteins, auracyanins A and B are both membrane-anchored electron transfer proteins involved in energy production, although they localize to different membrane sites, and their functions do not overlap; thus, each protein has distinct biological functions (see model in FIG. 11). Auracyanin A is proposed to be transported to an outer membrane to facilitate aerobic respiration, whereas auracyanin B is tethered to the inner membrane and has functions in photosynthesis and aerobic respiration. Unlike auracyanins A and B, azurins have been found only in non-photosynthetic proteobacteria and are transported to the periplasmic space without anchoring to the membrane. In contrast, the electron transfer complexes and subunits of ATP synthase in eukaryotic cells are localized at the mitochondrial inner membrane, similar to auracyanin B, which attaches to the bacterial inner membrane (FIG. 11). Our results suggest that the shared characteristics of some bacterial and mitochondrial proteins inherently enable bacterial proteins to target mitochondria (FIG. 8, FIG. 12). The differences in the subcellular localization and modes of action of these proteins despite their structural similarity may be explained by the discrepancy in their hydrophobicity/polarity. These differences may be why aurB, unlike aurA and p28, can localize to the mitochondria, bind to mitochondrial proteins, and subsequently induce caspase-mediated apoptotic cancer cell death independent of p53. Based on our concept and results described above, other mitochondria-targeting peptides can be identified from cupredoxins by larger screening studies.

### Example 8: High Levels of P. aeruginosa Azurin in the Sera of CF Patients

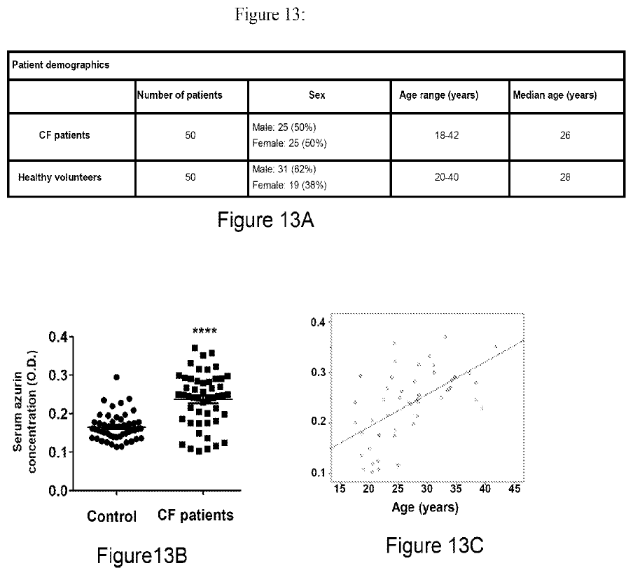

Based on the frequent colonization of CF patients with P. aeruginosa, the lower incidence of melanoma and breast cancer in CF than non-CF patients and previous descriptions of cancer-specific effects of azurin we hypothesized that azurin from P. aeruginosa plays a role in tumor development. To test our hypothesis that a bacterial protein, azurin, is detectable in human sera and that there is a significant elevation of azurin levels in CF patients, we first compared the serum levels of azurin in CF patients with chronic Pseudomonas infection to those in healthy volunteers with no disease (controls; see FIG. 13A) and found that azurin levels in CF patients were significantly higher than those in controls (FIG. 13B). The serum level of azurin was correlated with the age of CF patients (FIG. 13C). This clinical evidence showed that CF patients have significantly elevated levels of circulating azurin.

### Example 9: P. aeruginosa Azurin Secretion is Stimulated by Cancer Cells

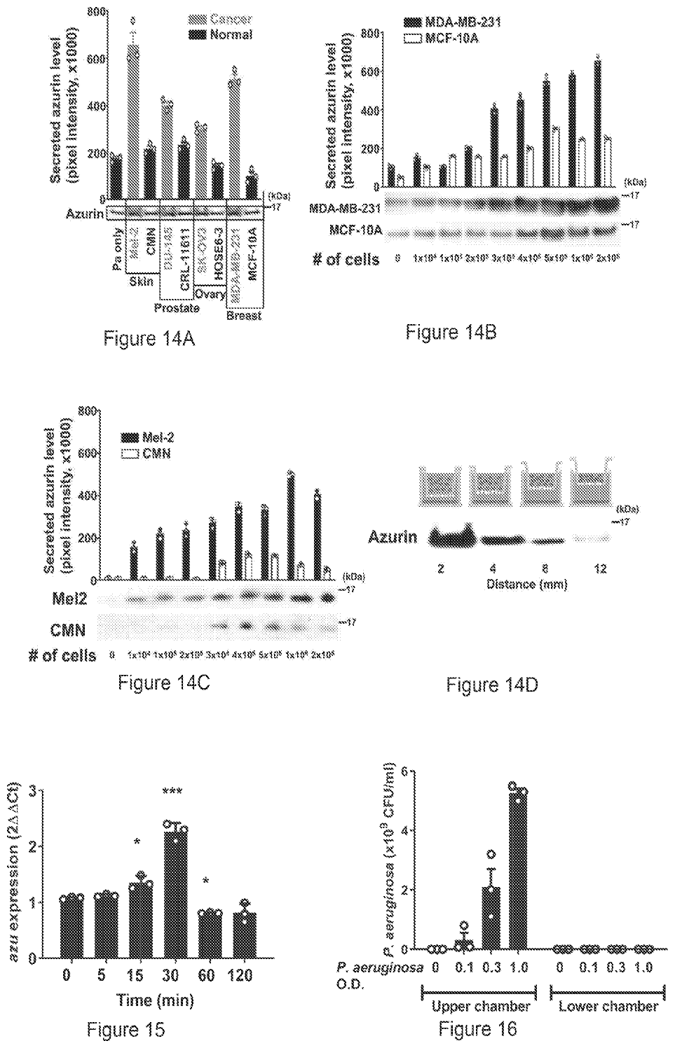

To investigate how host cells, either human cancer cells or their corresponding normal counterparts, affect P. aeruginosa azurin secretion, azurin secretion levels were measured in P. aeruginosa-host cell cultures. Azurin secretion was significantly higher, as were the transcription levels of the azurin-encoding gene azu, in the presence of various human cancer cell lines than in the presence of their normal counterparts (FIG. 14A, FIG. 15). The evidence that the incidence rates of melanoma and breast cancer are lower in CF patients and that these two types of cancers showed high contrast in azurin secretion in our experimental systems led us to focus on these cancers for the remainder of this study. Azurin secretion by P. aeruginosa was positively correlated with the number of human breast cancer (FIG. 14B) or melanoma (FIG. 14C) cells, and azurin secretion was significantly lower when P. aeruginosa was incubated with normal breast cells or benign nevus (congenital melanocytic nevus, CMN) cells (FIG. 14A-14C). These results suggest that P. aeruginosa preferentially secretes azurin in the presence of cancer cells and that this effect has a dose-dependent relationship with the population of cancer cells.

We next determined whether the bacteria-cancer interaction-mediated modulation of azurin secretion requires direct cell-to-cell contact between P. aeruginosa and cancer cells. Secreted azurin levels were highest at the shortest distance (2 mm) between the bacteria and cancer cells and quite low at distances greater than 12 mm, showing an inversely proportional correlation (FIG. 14D). Despite the lack of physical contact, azurin secretion was elicited, thereby indicating that a soluble factor secreted by Mel-2 cells may act as a stimulus. Changes in azurin levels were not due to P. aeruginosa contamination from top to bottom wells (FIG. 16). These results suggest the existence of at least one soluble agent originating from cancer cells that exerts a concentration-dependent stimulatory effect on azurin secretion.

### Example 10: Cancer Cells Secrete Aldolase a in Response to Azurin

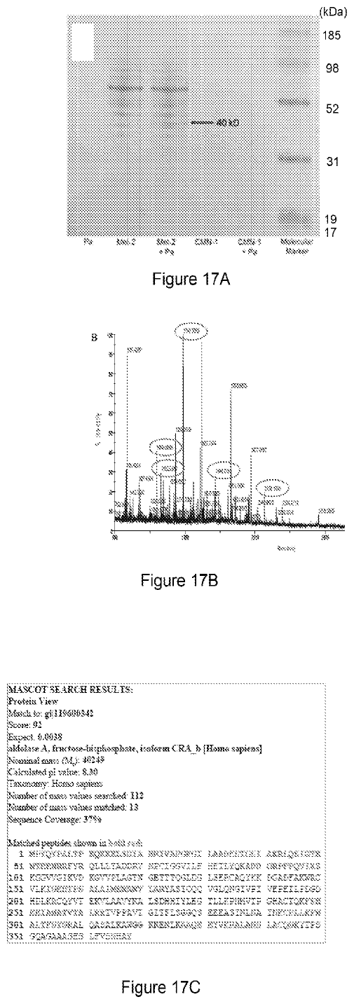

The significant stimulation of azurin secretion by malignant host cells compared to that by their normal counterparts suggested that these cell groups differed in the secretion of a host factor. To identify any proteins that were differentially secreted in the presence of P. aeruginosa, we used a mass spectrometry-based proteomic approach. We found that human aldolase A was released extracellularly into the culture medium when human cancer cells or their normal counterparts were co-incubated with P. aeruginosa (FIG. 17A-17C).

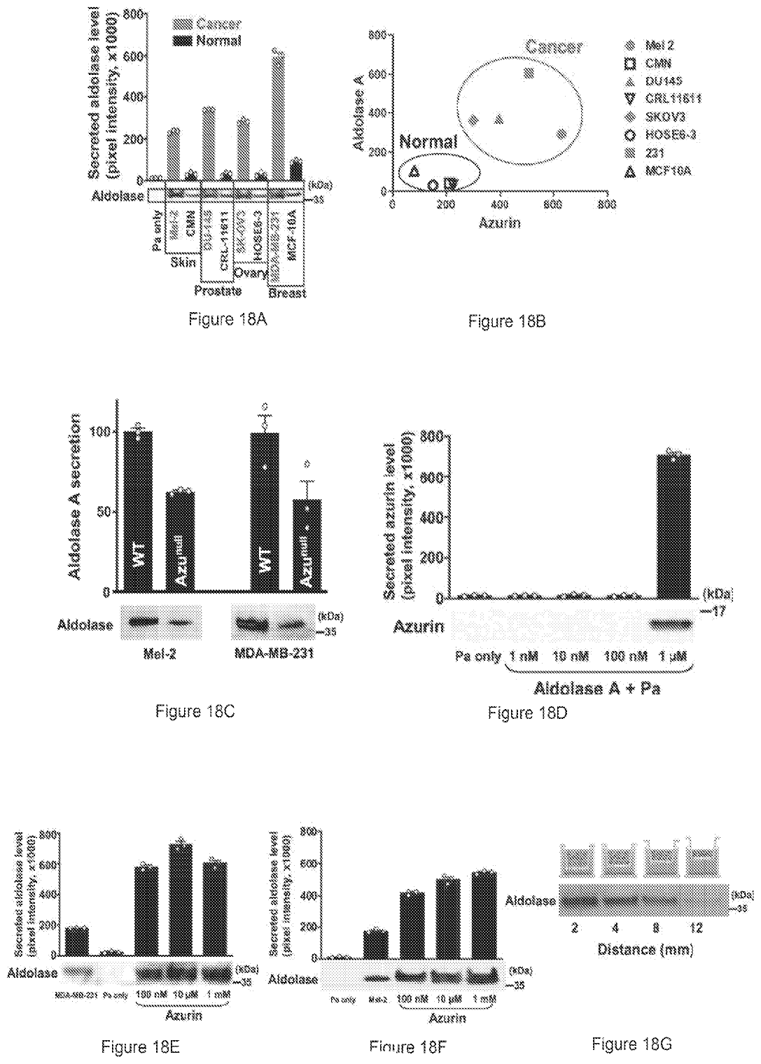

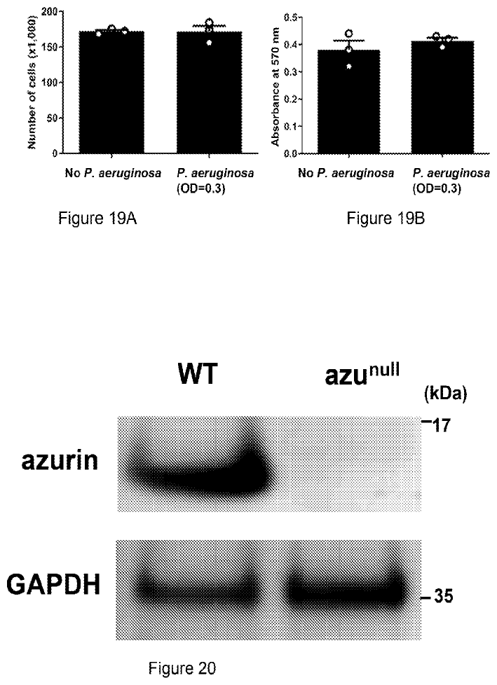

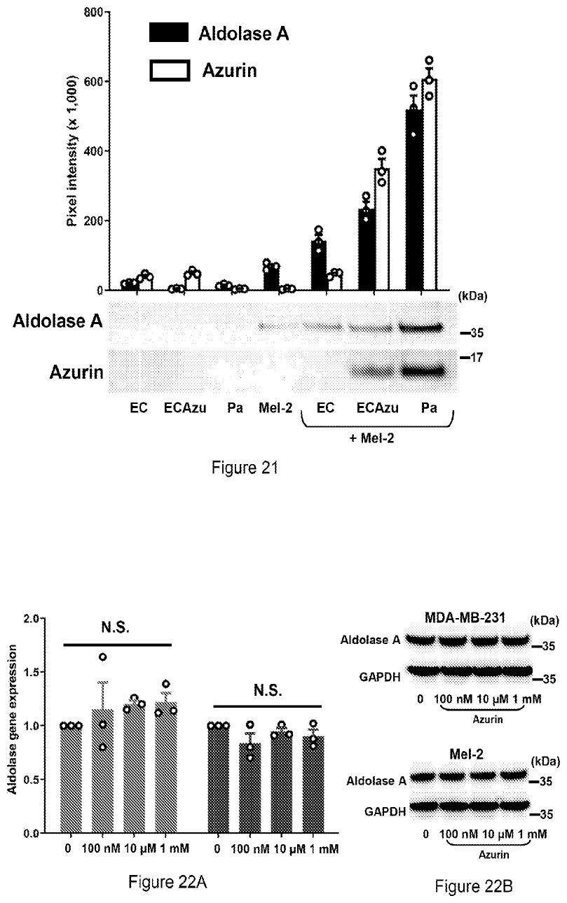

Aldolase, also called fructose-bisphosphate aldolase (FBA), is a glycolytic enzyme involved in the Embden-Meyerhof-Pamas glycolytic pathway and gluconeogenesis and is highly conserved in bacterial, archaeal, and eukaryotic organisms. Human aldolase A secretion by human cancer cells in the presence of P. aeruginosa was significantly higher than that by their normal counterparts (FIG. 18A). Changes in aldolase A secretion were not due to alteration of the cell growth rate or induction of toxicity in cancer cells (FIGS. 19A-19B). The secretion levels of aldolase A in culture medium were related to the secretion levels of azurin when human malignant and benign cells derived from various tumors and tissues were co-incubated with P. aeruginosa (FIG. 18B). To address the role of azurin on aldolase A secretion, three experimental approaches were taken: aldolase A secretion induced by (1) wild type (WT) and azu gene deleted mutant P. aeruginosa, (2) the non-pathogenic E. coli expressing azurin gene, and (3) recombinant azurin protein. First, aldolase A secretion was measured when cancer cells were co-incubated with WT and azu^{null} mutant P. aeruginosa. Aldolase A secretion induced by WT P. aeruginosa was significantly higher than azu^{null} mutant, but azu^{null} mutant can still induced aldolase A secretion from cancer cells (FIG. 18C, FIG. 20). Next, the interaction of the non-pathogenic E. coli laboratory strain JM109 (a K-12 derivative) transformed with the azurin-encoding gene with cancer cells was evaluated to determine whether the secretion of azurin and aldolase A is specific to the P. aeruginosa strain we used. E. coli expressing azu gene secreted a considerable amount of azurin in the presence of Mel-2 cells. Additionally, aldolase A secretion from Mel-2 cells was induced by azu-expressing E. coli (FIG. 21), suggesting that (1) azurin is a major inducer of aldolase A secretion, but not a sole inducer from P. aeruginosa, and (2) E. coli has a secretion mechanism similar to that of P. aeruginosa.

To further investigate the impact of human aldolase A on azurin secretion during the interaction of bacteria and human cancer cells, P. aeruginosa cells were cultured in the presence of purified human aldolase A (purity >95%), and azurin secretion was measured by western blotting. Exposure of P. aeruginosa to aldolase A induced azurin secretion (FIG. 18D). Conversely, when breast cancer and melanoma cells were cultured in the presence of purified azurin, these cancer cells secreted a considerable amount of human aldolase A into the culture medium (FIG. 18E, 18F) without increasing gene expression or intracellular levels of aldolase A, suggesting that aldolase A secretion induced by azurin occurs in a transcription-independent manner (FIGS. 22A-22B). Similar to the pattern of azurin secretion shown in FIG. 14D, the levels of secreted aldolase A were highest at the shortest distance (2 mm) between P. aeruginosa and cancer cells (FIG. 18G), that secreted azurin has a major role to induce aldolase A secretion, and their interaction is bidirectional and concentration density-dependent inter-communication between P. aeruginosa and cancer cells via azurin and aldolase A (FIG. 18).

### Example 11: Aldolase a Promotes P. aeruginosa Localization on Cancer Cells

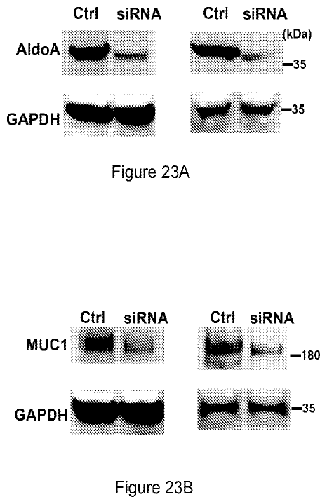

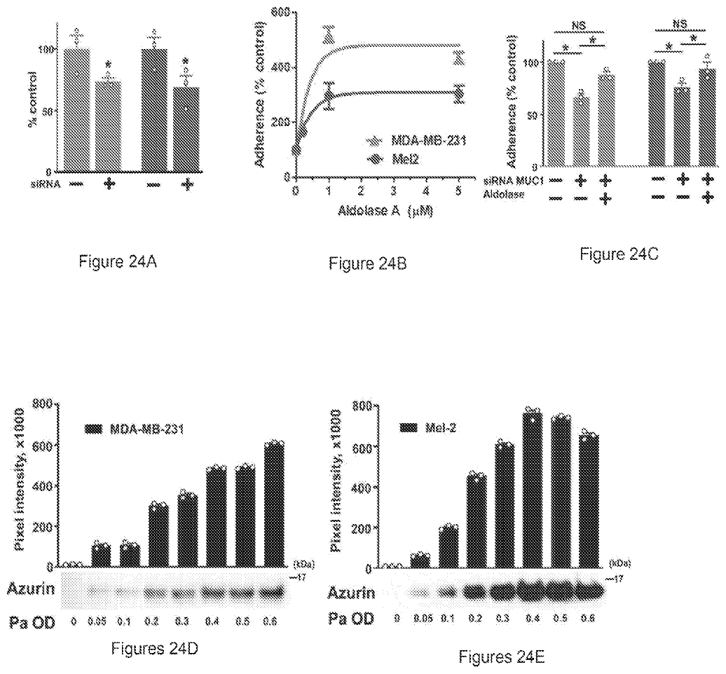

Although the cytosolic role of aldolase in glycolysis and gluconeogenesis has long been recognized, aldolase is reportedly also involved in host cell adhesion and biofilm formation in bacteria and parasites such as Streptococcus, Neisseria, Toxoplasma and Plasmodium. This prompted us to investigate whether aldolase A secreted by human cancer cells plays a similar biological role in the adherence of P. aeruginosa to cancer cells. First, we tested whether silencing aldolase A gene will alter the adherence of P. aeruginosa to cancer cells. The P. aeruginosa adhesion assay showed that siRNA-induced silencing aldolase A in MDA-MB-231 and Mel-2 cells (FIG. 23A) significantly reduced the adherence of P. aeruginosa (FIG. 24A).

Conversely, in the presence of purified human aldolase A, P. aeruginosa exhibited significantly increased adherence to cancer cells, and the increase was dose-dependent and saturable (>1 μM) (FIG. 24B). It has been reported that MUC1, an O-glycosylated membrane-tethered mucin on cancer cells, interacts with P. aeruginosa through flagellin44,45. Muc1−/−animals displayed ˜50% less adherence of P. aeruginosa in the lungs compared with Muc1+/+mice46. Hence, we tested the effect of siRNA-induced MUC1 silencing in cancer cells (FIG. 23B) by our P. aeruginosa adhesion assay. Silencing MUC1 in MDA-MB-231 and Mel-2 cells significantly reduced the adherence of P. aeruginosa (FIG. 24C). Moreover, when recombinant aldolase A was added to MUC1 silenced cancer cells, the adhesion rate of P. aeruginosa was similar to control values, suggesting that aldolase-mediated adherence of P. aeruginosa was, at least partly, independent of MUC1-mediated adhesion.

These results suggest that aldolase A induces P. aeruginosa adhesion and colonization in cancer cells. Although mucins have been suggested as the preferred sites for adherence and colonization of P. aeruginosa on host cells, P. aeruginosa can also bind to several other host proteins. These proteins include aldolase, and this binding is mediated by hydrophobic interactions.

### Example 12: Azurin Secretion is P. aeruginosa Density Dependent

The complexity of P. aeruginosa genome reflects evolutionary adaptation permitting it to thrive in diverse environments, including eukaryotic hosts, with which it has coexisted for millions ofyears. Pseudomonas spp. are renowned among prokaryotes for their complex quorum sensing (QS) systems that regulate biofilm formation. In general, bacterial QS of stimuli and responses correlates to population density. Based on our data, P. aeruginosa secretes low levels of azurin due to the presence of only a small population of either cancer cells (FIG. 14B, 14C) or P. aeruginosa (FIG. 24D, 24E). This finding suggests that a high concentration of aldolase A due to a large population of cancer cells enhances azurin secretion by increasing bacterial adherence and the density of the bacterial population. However, this mode of density sensing in P. aeruginosa is likely independent of the intercellular QS communication system since it has been previously shown that azurin is actively expressed in Pseudomonas mutants carrying mutations in the Gac/Rsm system that activates the QS machinery primarily by stimulating N-butanoyl-L-homoserine lactone (C4-HSL) production. Additionally, azurin expression is not regulated as a virulence factor in P. aeruginosa, as GacA is a major positive regulator of virulence in P. aeruginosa. Similar to the relationship between Burkholderia (previously considered members of the Pseudomonas genus) and plants, this QS-independent mechanism appears to play important roles in the P. aeruginosa-cancer interaction.

### Example 13: P. aeruginosa is Abundant in Primary Tumors, and its Product Azurin Inhibits Tumor Growth

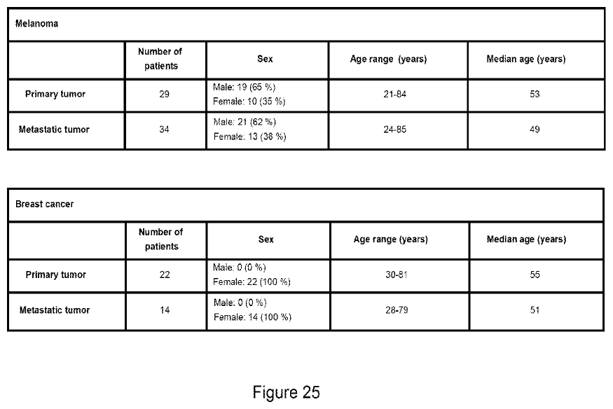

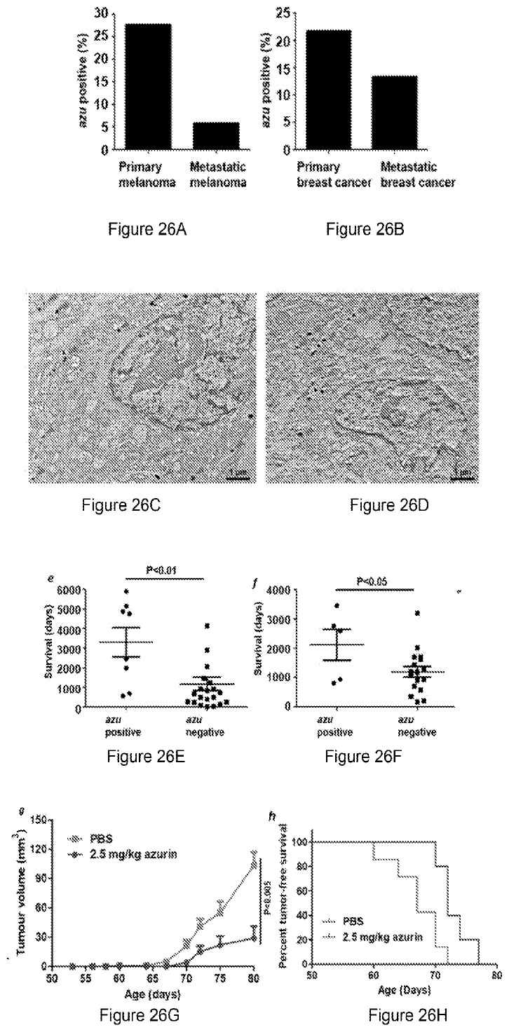

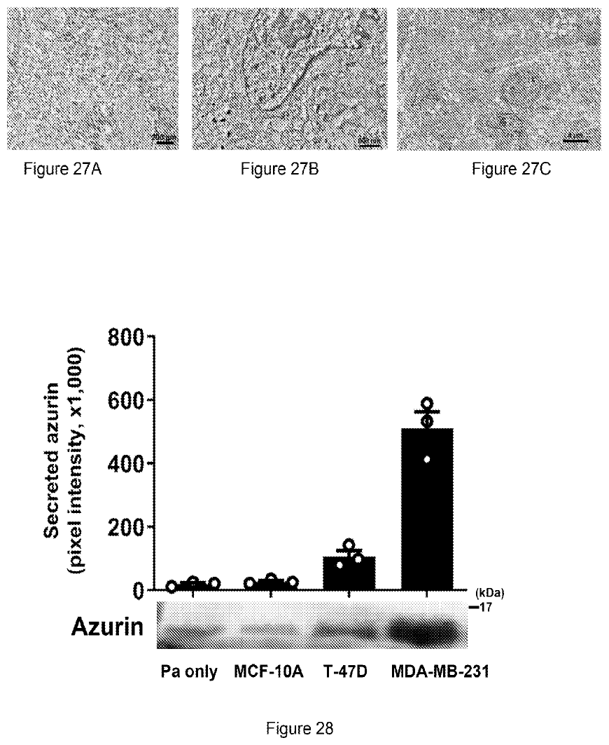

Our in vitro results indicated that P. aeruginosa and cancer cells communicate with each other through the secreted proteins azurin and aldolase A, leading to the localization of P. aeruginosa on cancer cells (FIGS. 14, 18, 24). This effect might be applicable to clinical settings. To measure P. aeruginosa localization in human melanoma and breast tumors, PCR with azu-specific primers was carried out. We included both primary and metastatic tumors, as their different characteristics may affect P. aeruginosa localization. Among tumors from patients with melanoma (primary: N=29, age range=21-84 years old; metastatic: N=34, age range=24-85 years old) and breast cancer (primary: N=22, age range=30-81 years old; metastatic: N=14, age range=28-79 years old) (FIG. 25), the P. aeruginosa azu gene was detectable in 27.6% of primary melanomas and 5.9% of metastatic melanomas (FIG. 26A). Primary breast cancers showed a higher positive rate for the azu gene than metastatic breast tumors (22.7% vs. 14.3%, FIG. 26B). The presence of P. aeruginosa and its product azurin was further confirmed by transmission electron microscopy (TEM) within azu-positive melanoma cells stained by an anti-P. aeruginosa antibody or anti-azurin antibody (FIG. 26C. 26D, FIG. 27A-27C). P. aeruginosa cells were found in the cytoplasm, and azurin was localized in both the cytoplasm and nucleus. This finding is consistent with those of our preclinical studies showing that azurin localizes in the nucleus. These results indicate that P. aeruginosa was detectable in human tumors, raising the possibility that, in some individuals, azurin-producing P. aeruginosa may affect the biological activity of tumors through bacterial-cancer interactions. To test this possibility, we compared survival between patients with azu-positive and those with azu-negative primary melanoma and breast cancer tumors. In both melanoma (FIG. 26E) and breast cancer (FIG. 26F), patients with azu-positive tumors had better overall survival times than patients with azu-negative tumors, suggesting that P. aeruginosa azurin positively affects the prognosis of patients, at least for those with melanoma or breast cancer.

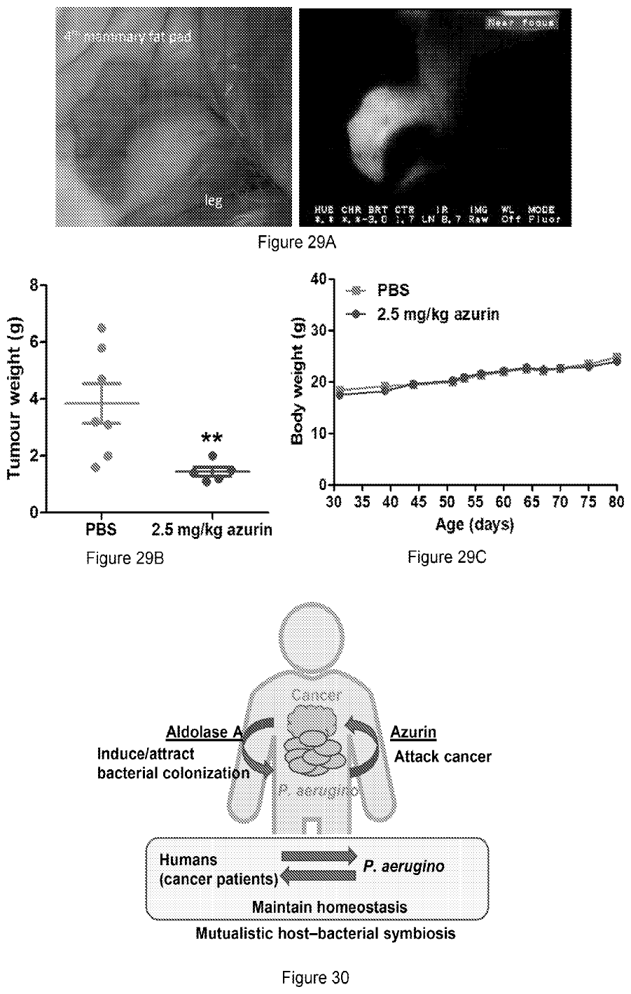

In this study, we used a transgenic mouse model that spontaneously develops mammary tumors to investigate the impact of P. aeruginosa azurin on cancer in vivo. Based on the critical role of azurin in P. aeruginosa-cancer interactions (FIGS. 14, 18, 24), purified P. aeruginosa azurin was injected into transgenic mice that spontaneously develop mammary tumors. An in vitro experiment indicated that P. aeruginosa azurin secretion was significantly higher in the presence of triple-negative MDA-MB-231 cells than in the presence of T-47D(ER+, PR+, Her2−) cells (FIG. 28); thus, triple-negative, p53 wt MMTV-PyMT transgenic mice were used. When these mice were exposed to azurin, NIR dye-conjugated azurin preferentially localized to spontaneously developed mammary tumors (FIG. 29A). Tumor growth and tumor weight were significantly inhibited without affecting body weight (FIG. 26G, FIG. 29B, 29C), and tumor-free survival was significantly extended in the azurin-treatment group (FIG. 26H). These findings suggest a new role for the bacterium P. aeruginosa in host defense via azurin production.

We demonstrate the bidirectional regulation of bacterial-cancer communication in relation to the potential for P. aeruginosa-secreted azurin to inhibit tumor growth. Human cancer cells upregulated aldolase A secretion in response to increasing proximity to P. aeruginosa/azurin, which correlated with enhanced P. aeruginosa adherence to the cancer cells. Our results also show that cancer patients had detectable P. aeruginosa and azurin in their tumors and exhibited increased overall survival when they did. Finally, our results suggest host-bacterial symbiotic mutualism acting as a diverse adjunct to the host defense system on tumor homeostasis.

This evidence suggests that some bacteria might be double-faced microbes in a symbiotic relationship that can both harm (parasitism) and benefit (mutualism) the host. Through evolution, P. aeruginosa has acquired abilities to both harm (parasitism) and benefit human health (mutualism) and adjusts its behavior depending on physiological/cellular conditions that can change depending on disease status. Our results suggest that P. aeruginosa can have a symbiotic relationship with humans characterized by both mutualism and parasitism. In its parasitic relationship, P. aeruginosa, an opportunistic pathogen, increases morbidity and mortality in patients with CF, while in its mutualistic relationship, P. aeruginosa can benefit human health by suppressing and attacking malignant cells through secretion of the bacterial protein azurin in hosts harboring malignant cells (FIG. 30). P. aeruginosa expressing azu was found in tumors of primary melanoma and breast cancer patients who did not receive chemotherapy before specimen collection. Furthermore, our preliminary data on human tumors indicated that azu-positive patients had longer overall survival times than azu-negative patients, suggesting that P. aeruginosa localized in tumors may positively influence cancer prognosis. We demonstrated that molecular determinants of host-bacterial mutualism act as diverse adjuncts to the host defense system through inter-kingdom signaling by evolutionarily conserved proteins, the bacterial cupredoxin azurin and human aldolase A. These data on a host-microbe interaction provided novel insight into the symbiotic strategies based on the microbial cupredoxin azurin in modulating host defense mechanisms, which occurred independent of immune system stimulation.

### Methods:

Peptide synthesis: Peptides were chemically synthesized (CS Bio, Menlo Park, CA) at >95% purity and mass balance. The sequences were as follows: aurA (auracyanin A aa 62-89): LVK GGE AEA ANI ANA GLS AGP AAN YLPA (SEQ ID NO:1), aurB (auracyanin B aa 61-88): LVN GGD DVA AAV NTA AQN NAD ALF VPPP(SEQ ID NO:2), and p28 (azurin aa 50-77): LST AAD MQG VVT DGM ASG LDK DYL KPDD (SEQ ID NO:3).

Cell lines and culture: Human cancer and noncancer (immortalized and nonimmortalized) cell lines were obtained from the American Type Culture Collection [prostate cancer (PC3, DU145 and LNCaP), normal prostate (CRL11611), breast cancer (MCF-7), ovarian cancer (SK-OV3 adenocarcinoma), lung cancer (A549)]. MDD2 breast cancer cells (p53 dominant-negative) were provided courtesy of Dr Andrei V. Gudkov, Roswell Park Cancer Institute. 20 All cell lines were cultured in MEM-E (Invitrogen) supplemented with 10% heat-inactivated fetal bovine serum (Atlanta Biological, Inc.), 100 units/mL penicillin, and 100 μg/mL streptomycin at 37° C. in 5% CO₂.

Cytotoxicity assay: The MTT assay was performed as we previously described (Cha et al., Cancer Research 2020, 80: 1615-1623).

Cellular membrane toxicity: LDH assays were performed as we previously described (Cha et al., Cancer Research 2020, 80: 1615-1623).

TEM: GNRs (Nanopartz Inc., CO) were conjugated in vitro with aurA and aurB according to the manufacturer's instructions. Briefly, NHS-functionalized non-spherical GNRs with a diameter of 25 nm×length of 73 nm were conjugated with 500 molar excess of aurA or aurB in 0.1 M borate buffer (pH 8.0) at room temp for 4 hr. The labeled peptides were washed with 1% PBS/0.1% Tween 20 at 9,000 rcf for 10 min and resuspended in PBS. After PC3 cells were exposed to aurA-GNRs or aurB-GNRs (300 μg/ml) for 16 hr, ˜1 mm3 cell culture samples were fixed in 4% phosphate-buffered glutaraldehyde, followed by washing samples with 2% sucrose in 0.1 M Sorensen's phosphate buffer at room temp. Samples were then treated with 1% osmium tetroxide in 0.1 M Sorensen's phosphate buffer for 1 hr at room temp. After washing with dH2O and dehydration with acetone, resin infiltration (EMBed 812 resin [EMS]) was performed as follows: 30 min, 2:1 mix of 100% acetone:resin; 30 min, 1:1 mix of 100% acetone:resin; 1 hr, 1:2 mix of 100% acetone:resin; and 1.5 hr, 100% resin. Samples were then placed in embedding molds and polymerized overnight at 60° C. Sections (60-nm thick) were made using an Ultratome and picked up on 200 mesh copper grids. TEM images of uranyl acetate- and lead citrate-stained samples were taken with a JEOL 1220 TEM.

Pull-down assay and protein identification: PC3 cells were washed twice with PBS, and the mitochondrial fraction was prepared with a Qproteome mitochondria isolation kit (Qiagen). Mitochondrial proteins were extracted with 10% n-dodecyl-β-D-maltoside (Mitochondrial Protein Immunoprecipitation kits, Sigma) and incubated with biotin-labeled p28, aurA or aurB for 16 hr at 4° C. The peptides were then preincubated with streptavidin-agarose beads for 4 hr. After washing, the beads were boiled, and the released proteins were subjected to SDS-PAGE analysis. A protein band of ˜35 kDa was subjected to microcapillary LC/MS/MS for protein identification (Harvard Medical School, Taplin Mass Spectrometry Facility). For IB, proteins were transferred to nitrocellulose membranes. After blocking with 5% BSA in TBST, the membranes were incubated with anti-ATP5C antibody at 1:25,000 in 5% BSA/TBST (Abcam) for 16 hr at 4° C. The secondary antibody was applied (anti-goat IgG-HRP; Santa Cruz Biotechnology). The signal was detected using enhanced chemiluminescence (ECL).

Flow cytometric analyses: Three prostate cancer cell lines and a normal cell line were treated with Ptxl at 1 nM, p28 at 50 μM, aurA at 0.5-50 μM or aurB at 0.5-50 μM. After 48 hr, annexin-V apoptosis assays (Thermo Fisher Scientific) were conducted to detect apoptotic cells. At least 10,000 cells in each case were analyzed by FACS (RRC, UIC).

JC-1 dye (Invitrogen) was used to determine mitochondrial membrane potential. PC3 cells were exposed to 1, 10 and 100 μM aurA or aurB. After washing with PBS, trypsinized PC3 cells were incubated with JC-1 dye and analyzed by FACS.

Caspase assay: Similar to the procedure for the MTT assays, PC3 cells were exposed to aurB in the presence or absence of the specific caspase inhibitor Z-DEVD-FMK.

Xenograft animal model: Human prostate cancer cells (PC3) were injected s.c. into the right flanks of 5- to 6-week-old male athymic mice 78. When tumors reached 5 mm in diameter, animals were randomized into control and treatment groups. The dosages were as follows: Ptxl (10 mg/kg, once a week), p28 (10 mg/kg daily), aurA (10 mg/kg daily), and aurB (5 mg/kg daily), i.p. The results are shown as the mean+SE (N=5). All animals were weighed twice a week. At necropsy, tumors were dissected and weighed. Statistical comparisons were performed by one-way analysis of variance (ANOVA) (control vs treatment). Tumor samples were fixed overnight in 10% buffered formalin. Fixation was followed by dehydration, clearing and infiltration. Samples were then embedded in paraffin, and 4 μm sections were cut on a microtome. Ki67 (LabVision) staining was performed on the tissue sections using a Vector Vectastain Elite ABC Kit. The TUNEL assay was performed on the sections using a Millipore Sigma ApopTag® Peroxidase In Situ Apoptosis Detection Kit.

P. aeruginosa and human cell lines: P. aeruginosa strain 8822 isolated from the sputum of a CF patient is a generous gift from Dr. Ananda M. Chakrabarty. Human cell lines of prostate cancer (DU-145), normal prostate (CRL-11611), ovarian cancer (SK-OV3), normal ovary (HOSE6-3); breast cancer (MCF-7, T-47D, MDA-MB-231), normal breast (MCF-10A) were purchased from the American Tissue Culture Collection (ATCC, VA). Human melanoma (UISO-Mel-2) and CMN cell lines were developed in our laboratory as described.

ELISA: CF subjects were patients followed at the Adult CF Clinic at Emory University who had signed informed consent to provide blood samples in accordance with the Emory University IRB (Emory #00042577). Control sera from no-disease volunteers were obtained from Discovery Life Sciences (Los Osos, CA). Serum samples (N=50 in each group) in carbonate bicarbonate buffer (pH 9.4) were used to coat 96-well plates (MaxiSorb, Thermo Fisher) in triplicate. Standard curves as internal controls were generated by using purified azurin75 to coat 96-well plates. Polyclonal rabbit anti-azurin antibody1,2 and alkaline phosphatase-conjugated secondary anti-rabbit antibody (SigmaMillipore) were used to detect azurin.

Growth media: All human cell lines were maintained in MEME with 10% FBS except for MCF-10A which was maintained in DMEM with 10% FBS. Luria-Bertani medium was used to grow P. aeruginosa before transfer to the experiment's medium of 0.5% Minimal Glucose Medium (MGM) without any antibiotics.

P. aeruginosa quantitation: This was conducted using the turbidimetry method after the establishment of the standard growth curve of P. aeruginosa in 0.5% MGM. An optic density (OD) of 0.3 correlated with the mid-log phase of the bacterial growth and was chosen as the standard OD at 600 nm for the entire experiment.

Expression and purification of azurin: Cloning, expression of azu gene in E. coli and purification of recombinant azurin were described previously1.

P. aeruginosa and human cancer cells co-incubation: P. aeruginosa strain 8822 were grown overnight in LB medium at 37° C. then transferred at 1:100 v:v into 0.5% MGM without adding antibiotics. The OD of the medium was measured until the goal OD for the experiment was reached. Human cells were grown in the appropriate medium as reported above. Cells were trypsinized and counted using a Coulter Counter® Cell and Particle Analyzer. The needed number of cells was washed 2× with PBS and finally suspended in 0.5% MGM. For direct co-incubation assays, human cells and P. aeruginosa were co-incubated for 30 min at a concentration of 500,000 human cells/ml and P. aeruginosa at an optical density (OD)=0.3. The needed number of cells was transferred based on the specifics of each experiment. For the indirect co-incubation assays, Corning Transwell® polyester membrane cell culture plates and inserts (TC-treated, sterile 24 mm Transwell with 0.4 μm pores, SigmaMillipore) were used. Human cells were immobilized in the lower compartment (well) whereas bacteria were incubated in the upper compartment (insert) at 37° C. for 30 min with a semi-permeable membrane of 0.4 μm pores separating the two compartments. Wild type (WT) P. aeruginosa PAOI and its azu^{null} mutant cells were used for aldolase secretion assay. Mutant P. aeruginosa was maintained on LB agar plates containing spectinomycin. Other experimental conditions were the same as above.

Protein extraction: Comparative analyses of secreted proteins in media were performed as described previously. The same volume of medium was collected from cell suspensions (direct co-incubation assays) or the lower compartment well (indirect co-incubation assays) after incubation and centrifuged at high speed (12,000×g). The supernatant was then filtered using an Amicon@0.22 μm pore filter. The equal volume of trichloroacetic acid (TCA; final concentration of 20%) was added to the supernatant and incubated on ice for 30 min. After spinning at 18,000×g, TCA was decanted, and 100% acetone precipitation was applied twice with 5 min incubation. At the final step, acetone was decanted, and protein pellets were allowed to dry at room temperature for 15 min and resuspended in PBS. The equal volume of each sample was loaded per lane.

Western blotting: After running the NuPAGE, proteins were transferred to PVDF membranes (BioRad Laboratories Inc, Hercules, CA) which were blocked in SuperBlock (T20 TBS buffer, Thermo Fisher) for one hour. For secreted proteins, equal loading was confirmed by Ponceau staining of the membranes79. The PVDF membranes were incubated in a polyclonal rabbit anti-azurin antibodyl (1:5000) or a monoclonal mouse anti-aldolase A (1:200, Santa Cruz Biotechnology, TX) at 4° C. overnight. For cell lysates, GAPDH was used as a loading control (Novus Biologicals). The secondary antibody was applied (1:1000, polyclonal goat anti-rabbit IgG-HRP; Santa Cruz Biotechnology). The signal was detected using enhanced chemiluminescence (ECL, Thermo Fisher Scientific). Quantitative analysis of the bands was performed with a computer-assisted imaging densitometer (UN-SCAN-IT gel version 5.1).

Host cell adhesion assay: The assays were performed essentially as described before38. Cancer cells were co-incubated with P. aeruginosa 8822 in 96-well plates in the presence or absence of various concentrations of aldolase A for 2 h. Monolayers of cancer cells were extensively washed with PBS to remove unbound P. aeruginosa. Monolayers were homogenized in 0.1% saponin (SigmaMillipore) in PBS. P. aeruginosa were enumerated by serial dilution of the homogenized suspensions and subsequent determination of colony-forming units (CFU/ml) by plating on LB agar plates. Control values of CFU/ml expressed as 100%. siRNA-induced silencing of aldolase and MUC1 genes in MDA-MB-231 and Mel2 cells as conducted as described previously80,81. Briefly, SMARTpool human ALDOA, MUC1, and non-targeting siRNA pool (non-targeting siRNAs) (Dharmacon, PA) were used as siRNA targeting aldolase A, MUC1, and negative control, respectively. MDA-MB-231 and Mel2 cells were seeded in 96-well plates, cultured to 80-90% confluence, transfected with 120 nM siRNAs using FuGENE HD (Promega) according to the manufacturer's protocol for 48 h, and used for host cell adhesion assays. Knockdown of ALDOA and MUC1 by siRNA transfection was examined by western blot analyses using monoclonal mouse anti-aldolase A and MUC1 antibodies (1:200, Santa Cruz Biotechnology).

Detection of azurin gene in human tumors: Tumor samples of breast cancer and melanoma were collected at University of Illinois at Chicago. All patients included in the analysis were diagnosed with either breast cancer or melanoma. Supplementary FIG. 10 contains relevant patient information with similar age range and median age. The detection of azu gene in the human tumors was performed by PCR with azu-specific primers; 5′-CAGTTCACCGTCAACCTGTCC-3′(SEQ ID NO: 5) and 5′-TGGTGTGGGCGATGACACG-3′(SEQ ID NO: 6). Human GAPDH was also amplified as a loading control; 5′-AACGGGAAG CTTGTCATCAA-3′(SEQ ID NO: 7) and 5′-TGGACTCCACGACGTACTCA-3′(SEQ ID NO: 8). We used ultraclean instruments, kits, and reagents to minimize and control for contamination. DNeasy Blood & Tissue Kits (Qiagen) were used to prepare template DNA. The amplified PCR products were confirmed as a single band on 2% agarose gels. Products were purified by using QIAquick PCR Purification kits (Qiagen) and sequenced with an ABI Prism 3700 DNA analyzer. The Fisher's Exact Test was applied to determine if the positive rates are significantly different between the subjects with the primary and the metastatic tumors.

P. aeruginosa and azurin visualization by TEM: Frozen tumors were processed for immunogold transmission electron microscopy in accordance with a published method82,83 with minor modifications. Tumor samples along with their survival data were obtained from patients who had signed informed consent (IRB #H-96-772). The frozen samples were diced into 1×1 mm cubes on dry ice then fixed by incubating them in PBS-containing 4% paraformaldehyde and 0.75% glutaraldehyde for 72 h at 4° C. The samples were thoroughly washed with PBS then were incubated with PBS-containing 0.1% saponin for 1 h at room temperature on a rotary mixer (Ted Pella, Inc, Redding, CA). After the samples were thoroughly washed with PBS, they were incubated with PBS-containing 5% bovine serum albumin (BSA), 0.1% cold water fish skin gelatin (CWFS), and 5% normal goat serum (NGS) (Goat Gold Conjugate Blocking Solution, Electron Microscopy Sciences, Hatfield, PA) for 1 h at 4° C. The samples were thoroughly washed with PBS-containing 0.1% acetylated bovine serum albumin (BSA-c) (Aurion BSA-c, Electron Microscopy Sciences) then incubated with either mouse anti-Pseudomonas aeruginosa antibody (clone B11, 1:50 dilution, Thermo Fisher Scientific, Waltham, MA) or rabbit anti-azurin antibody (1:100 dilution)1 for 48 h at 4° C. After extensive washing, the samples were incubated with 0.1% BSA-c/PBS-containing either goat anti-mouse or goat anti-rabbit secondary antibody for 24 h at 4° C. To visualize each specific primary antibody, these antibodies were labeled with 10 nm gold particles (1:50 dilution, Electron Microscopy Sciences). The samples were washed with PBS-0.1% BSA-c and deionized water then incubated with deionized water containing 2.5% glutaraldehyde (Electron Microscopy Sciences) for 1 h at room temperature. After extensive washing with deionized water, the sections were fixed with deionized water containing 0.5% osmium tetroxide and 1.5% potassium ferricyanide (Electron Microscopy Sciences) for 15 min in the dark. Next, the samples were dehydrated by incubation in an ascending series of ethanol (25, 50, 75, 95, 100%, Electron Microscopy Sciences) followed by incubation in a 1 to 1 ratio of 100% ethanol to epoxy resin (comprised of a mixture of EMbed 812, nadic methyl anhydride, dodecenyl succinic anhydride, and 2,4,6-Tris(dimethylaminomethyl)phenol, Electron Microscopy Sciences) for 12 h at room temperature on a rotary mixture (Ted Pella, Inc). The samples were incubated with 100% epoxy resin for 12 h at room temperature on a rotary mixer (Ted Pella, Inc). The epoxy resin was changed and the samples were incubated for 2 h at room temperature on a rotary mixer. The samples were placed into flat embedding molds filled with epoxy resin then allowed to polymerize at 70° C. for 24 h. Ultrathin sections (70 nm) were cut with an ultramicrotome (EM UC7, Leica Microsystems, Buffalo Grove, IL), mounted on formvar- and carbon-coated 200 mesh copper grids (Electron Microscopy Sciences) then stained with filtered 1% uranyl acetate prior to imaging. Samples were imaged with a Philips CM 120 transmission electron microscope (TSS Microscopy, Hillsboro, OR) equipped with a BioSprint sixteen-megapixel digital camera (Advanced Microscopy Techniques, Woburn, MA).

Patients' survival: Survival of each patient of primary melanoma and breast cancer was measured from the date of diagnosis until death from any cause or last follow-up. The experiments were based on randomized trials and the investigators were blinded to the assignments in the experiments and outcome evaluations. Two independent sample tests were performed for comparing the survival days of azu gene positive and negative groups. The Shapiro-Wilk's test showed that normality was severely violated in data of melanoma patients.

The Wilcoxon's rank sum test for the medians in the melanoma survival data was chosen due to its robustness to the deviation from normality. For breast cancer patients, Shapiro-Wilk's test showed that the survival days in both azu gene positive and negative groups can be well-modeled by a normal distribution. As equal variances between the two groups were reasonable and was confirmed by a F test, a pooled t test on means was performed to compare the survival days between azu gene positive and negative groups. Data were analyzed using R (v.4.0.2) and RStudio (v.1.3.1093).

Effect of azurin on transgenic mice: Hemizygous MMTV-PyMT (mouse mammary tumor virus-polyoma middle tumor-antigen) female mice were obtained from The Jackson Laboratory. Since MMTV-PyMT mice develop spontaneous mammary tumors that closely resemble the progression and morphology of human breast cancers, the mouse model is widely used and well characterized84. Mice at 4-weeks old were randomized into control (N=7) and azurin-treatment (N=5) groups. Control animals received PBS and azurin-treatment animals received 2.5 mg/kg of azurin in sterile PBS i.p. 3× week for nearly two months (80 days old). Based on azurin levels in CF patients' sera, the highest level of azurin was 32 μg/ml. In order to reach this level of azurin in mouse (25 g-body weight, 2 ml total blood volume), azurin does at 2.5 mg/kg was determined. Tumor volume and body weights were determined three times weekly. All tumors were excised and weighted at the end of the study. All experiments were approved by University of Illinois at Chicago (UIC) Institutional Animal Care and Use Committee (IACUC) and conformed to the guidelines set by United States Animal Welfare Act and the National Institutes of Health.

Statistics and reproducibility: The paired student's t test the one-way analysis of variance were used for comparisons. P value <0.05 was considered significant. Number of replicates (N) can be found in the figure legends. Data were analyzed using Graphpad Prism software (ver. 8), R (ver.4.0.2), and RStudio (ver.1.3.1093).

RNA extraction and real-time PCR for azurin expression: RT-PCR was performed with 1 μg of total RNA from control and treated samples using the SuperScript First-Strand Synthesis System for real-time reverse transcriptase polymerase chain reaction (RT-PCR) (Invitrogen, Carlsbad, CA) and SYBR® Green PCR Master Mix (Applied Biosystems, Foster City, CA). Software from Integrated DNA Technologies (Coralville, IA) was used to design primers. The reaction was conducted as a two-step RT-PCR procedure using RT-PCR reagents kits from Invitrogen, and ABI 7500FAST Sequence Detection System (Applied Biosystems, Foster City, CA). After the last amplification cycle, the PCR products were denatured to generate a dissociation curve. The fluorescent signal was detected and analyzed by ABI Prism® 7500 Sequence Detection Software (Applied Biosystems, Foster City, CA). The threshold cycles (Ct) were calculated by the software and used to determine relative expression of genes.

Protein identification by Mass spectrometry: Mel-2 and P. aeruginosa 8822 were co-incubated using the Transwell system and secretions were concentrated using Amicon Centriprep YM-3 centrifugal filters (Thermo Fisher Scientific). The concentrated samples were run on SDS-PAGE. The gels were stained with Coomassie Brilliant Blue R-250 (CBB) (0.1% CBB R-250, 20% methanol, 0.5% acetic acid) and destained for an hour (BioRad). The Mel-2 and P. aeruginosa sample demonstrated an additional band when compared to a Mel-2 only sample. The additional protein band was cut from the gel. In-gel tryptic digestion and matrix-assisted laser desorption/ionization time-of-flight (MALDI-TOF) mass spectrometry of tryptic peptides was performed as follows. Gel plugs were washed in 50% acetonitrile, reduced of sulfide bonds in 60 mM DTT, alkylated of free sulfhydryl groups in iodoacetamide, 50 mM ammonium bicarbonate (pH 8.0) and 5 mM EDTA, and then incubated in trypsin [in 50 mM ammonium bicarbonate (pH8.0) solution at a concentration of 2 μg/100 μl] overnight. For MALDI-TOF, residual peptides were extracted, spotted onto a MALDI-TOF target, and analyzed by a positive-ion reflector mode with delayed extraction over the m/z range 700-4000 using a Voyager DE-PRO Mass Spectrometer (Applied Biosystems, Foster City, CA) equipped with a nitrogen laser. Spectra were externally and internally calibrated. Peptide mass results were used to identify the proteins using the MASCOT Peptide Fingerprint link.

Cell count and MTT assays: Growth of Mel-2 cells after co-incubation with P. aeruginosa 8822 was assessed by counting the number of cells at the desired time point and comparing the numbers to the initial cell count. The cells were plated in 24-well cell culture plates at a density of 4×105 cells/well in MEME supplemented with FBS, L-glutamine and non-essential amino acids. They were incubated at 37° cina5% CO humidified atmosphere and allowed to adhere ovemight. P. aeruginosa (OD=0.3) was co-cultured with Mel-2 for 30 minutes. Following the incubation, P. aeruginosa was aspirated, Mel-2 were washed with PBS, trypsinized, made into a single cell suspension, diluted with Isoton II Diluent, and counted in triplicates using the Coulter Counter Cell and Particle Analyzer. The total number of cells was obtained by considering the dilution factor and the total volume of the cell suspension from each well. The potential antiproliferative effects of P. aeruginosa 8822 on Mel-2 viability were also evaluated using the 3-[4,5-dimethylthiaolyl]-2,5-diphenyl-tetrazolium bromide (MTT) assay (TACS MTT cell proliferation assay kits, Trevigen, Gaithersburg, MD). Cells were seeded in 96 well plates at a density of 5,000 cells/well. Cell viability was analyzed after co-incubation with P. aeruginosa for 30 minutes. Following co-incubation with P. aeruginosa, Mel-2 were incubated with MTT tetrazolium reagent for 2 h at 37° C., and the absorbance of formazan was then measured at 570 nm. Each treatment was performed in triplicates, and the percent of cell growth inhibited was calculated by comparison of the absorbance readings of the control (Mel-2 only) versus Mel-2 co-incubated with P. aeruginosa.

Gene expression and intracellular levels of aldolase A. MDA-MB-231 and Mel-2 were treated with azurin for 30 min. Total RNA was extracted from cancer cells and cDNAs was generated by using High-Capacity cDNA Reverse Transcription Kits (Thermo Fisher Scientific). Conditions of reverse transcription were as follows: 25° C. for 10 min, 37° C. for 120 min, 85° C. for 5 min. Expression levels of aldolase A was determined by RTPCR using SYBR Green assay following the manufacturer's instructions [PowerUp SYBR Green Master Mix (Life Technologies, USA)]. The relative expression of aldolase A was calculated using the comparative Ct method. Data (N=3) was normalized with β-actin gene as a housekeeping gene. ΔCt=CtaldoseA-Ctβ-actin. The changes of treatment in aldose A signal relative to the total amount of cDNA were expressed as ΔΔCt=Ct Treatment−Ct Control. Relative changes in treatment were then calculated as 2-ΔΔCt. The following primer sequences were used: Aldolase A: forward 5′-CGG GAA GGA GAA CCT G-3′(SEQ ID NO: 9) and reverse 5′-GAC CGC TCG GAG TGT ACT TT-3′(SEQ ID NO: 10); and β-actin: forward 5′-ACT GGA ACG GTG AAG GTG AC-3′(SEQ ID NO: 11) and reverse 5′-AGA GAA GTG GGG TGG CTT TT-3′(SEQ ID NO: 12). Real-time PCR was performed under the following conditions: 50° C. for 2 min, 95° C. for 2 min; 40 cycles at 95° C. for 15 sec and 60° C. for 1 min; and 95° C. for 15 sec, 60° C. for 1 min and 95° C. for 15 sec.

NIR-imaging of transgenic mice: Near infrared red fluorescent dye (IR800, Licor, NE) was conjugated to azurin according to the manufacturer instructions. Transgenic mice with spontaneously developed mammary tumours received NIR dye conjugated azurin at 5 mg/kg once i.p. After 24 h, specific fluorescence signal at 800 nm was recorded by the PDE-neo® NIR camera system (Hamamatsu photonics, Mitaka-USA).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An isolated auracyanin peptide that modulates mitochondrial mediated apoptotic pathways within a eukaryotic cell.

2. The auracyanin peptide of claim 1, wherein the peptide is 20-35 amino acids in length.

3. The auracyanin peptide of claim 2, wherein the peptide is 28 amino acids in length.

4. The auracyanin peptide of claim 2, wherein the peptide forms α-helical and β-sheet motifs in solution.

5. The auracyanin peptide of claim 2, wherein the auracyanin is a C. aurantiacus protein.

6. The auracyanin peptide of claim 2, wherein the peptide is amino acids 62-89 of an auracyanin A protein.

7. The auracyanin peptide of claim 2, wherein the peptide is amino acids aa 61-81 of an auracyanin B protein.

8. The auracyanin peptide of claim 2, wherein the peptide localizing to the mitochondrial membrane of the eukaryotic cell and binding to the gamma subunit of a human mitochondrial ATP synthase protein.

9. The auracyanin peptide of claim 8, wherein the peptide targets mitochondrial mediated apoptotic pathways within cancer cells but not healthy cells.

10. The auracyanin peptide of claim 1, wherein the peptide has a sequence that is 96% identical to SEQ ID NO: 1 or 2.

11. The auracyanin peptide of claim 1, wherein the peptide has a sequence that is SEQ ID NO: 1 or 2.

12. The auracyanin peptide of claim 2, wherein the peptide has a pI of about 3.42 and a molecular weight of about 2400-2700 Da.

13. The auracyanin peptide of claim 2, wherein the peptide is non-toxic to the eukaryotic cell and penetrates the eukaryotic cell.

14. The auracyanin peptide of claim 2, wherein the peptide produces mitochondrial membrane potential loss in a dose dependent manner.

15. A method of modulating mitochondrial mediated apoptotic pathways within a eukaryotic cell comprising:
administering to a eukaryotic cell a therapeutically effective amount of an auracyanin peptide that is amino acids aa 61-81 of an auracyanin B protein,
wherein the auracyanin peptide induces produces mitochondrial membrane potential loss causing the induction of a caspase-mediated apoptotic pathway.

16. The method of claim 15 wherein the auracyanin peptide induces caspase-mediated apoptotic cell death in cancer cells but not healthy cells.

17. The method of claim 15 wherein the mediated cell death induced by the auracyanin peptide is independent of a tumor suppressor protein p53 pathway.

18. A method of administering to a subject in need of treatment for a cancer a therapeutically effective amount of an auracyanin peptide comprising:
providing an amount of auracyanin peptide effective to enhance mitochondrial mediated apoptotic pathways within a cancer cell in a pharmaceutically acceptable form;
administering the effective amount of auracyanin peptide to the subject,
wherein the auracyanin peptide will induce caspase-mediated apoptotic pathways in cancer cells.

19. The method of claim 19 wherein the auracyanin peptide is amino acids aa 61-81 of an auracyanin B protein.

20. The method of claim 19, wherein bringing P. aeruginosa in proximity to a cancer so that bidirectional communication between the P. aeruginosa and cancer cells results a P. aeruginosa secreted azurin inhibiting cancer growth.
